SEQUENCE LISTING (27 sequences)

SEQ ID NO: 1 - DNA, 30 bp, Artificial Sequence
  Synthetic Construct
gtttttcggctattcccaatagccgttttg

SEQ ID NO: 2 - protein, 8 aa, Artificial Sequence
  Synthetic Construct
  MISC_FEATURE (2)..(2): Xaa is Val or Ile
  MISC_FEATURE (4)..(4): Xaa is any amino acid
DXEXNPGP

SEQ ID NO: 3 - protein, 9 aa, Artificial Sequence
  Synthetic Construct
GDVESNPGP

SEQ ID NO: 4 - protein, 9 aa, Artificial Sequence
  Synthetic Construct
GDIEENPGP

SEQ ID NO: 5 - protein, 7 aa, Artificial Sequence
  Synthetic Construct
VEPNPGP

SEQ ID NO: 6 - protein, 7 aa, Artificial Sequence
  Synthetic Construct
IETNPGP

SEQ ID NO: 7 - protein, 9 aa, Artificial Sequence
  Synthetic Construct
GDIESNPGP

SEQ ID NO: 8 - protein, 9 aa, Artificial Sequence
  Synthetic Construct
GDVELNPGP

SEQ ID NO: 9 - protein, 9 aa, Artificial Sequence
  Synthetic Construct
GDIETNPGP

SEQ ID NO: 10 - protein, 8 aa, Artificial Sequence
  Synthetic Construct
GDVENPGP

SEQ ID NO: 11 - protein, 9 aa, Artificial Sequence
  Synthetic Construct
GDVEENPGP

SEQ ID NO: 12 - protein, 9 aa, Artificial Sequence
  Synthetic Construct
GDVEQNPGP

SEQ ID NO: 13 - protein, 7 aa, Artificial Sequence
  Synthetic Construct
IESNPGP

SEQ ID NO: 14 - protein, 9 aa, Artificial Sequence
  Synthetic Construct
GDIELNPGP

SEQ ID NO: 15 - protein, 9 aa, Artificial Sequence
  Synthetic Construct
HDIETNPGP

SEQ ID NO: 16 - protein, 9 aa, Artificial Sequence
  Synthetic Construct
HDVETNPGP

SEQ ID NO: 17 - protein, 9 aa, Artificial Sequence
  Synthetic Construct
HDVEMNPGP

SEQ ID NO: 18 - protein, 9 aa, Artificial Sequence
  Synthetic Construct
GDMESNPGP

SEQ ID NO: 19 - protein, 9 aa, Artificial Sequence
  Synthetic Construct
GDVETNPGP

SEQ ID NO: 20 - protein, 9 aa, Artificial Sequence
  Synthetic Construct
GDIEQNPGP

SEQ ID NO: 21 - protein, 8 aa, Artificial Sequence
  Synthetic Construct
DSEFNPGP

SEQ ID NO: 22 - protein, 9 aa, Artificial Sequence
  Synthetic Construct
  MISC_FEATURE (1)..(1): Xaa is absent or Gly or His
  MISC_FEATURE (2)..(2): Xaa is absent or Asp or Gly
  MISC_FEATURE (3)..(3): Xaa is Asp or Val or Ile or Ser or Met
  MISC_FEATURE (5)..(5): Xaa is any amino acid
XXXEXNPGP

SEQ ID NO: 23 - RNA, 9 nt, Artificial Sequence
  Synthetic Construct
  misc_feature (3)..(3): r is a or g
ccrccaugg

SEQ ID NO: 24 - RNA, 96 nt, Artificial Sequence
  Synthetic Construct
ggcucaucucgacaagaggcggcaguccucaguacucuuacucuuuucuguaaagaggagacugcuggacucgccgcccaaguucgagcaugagcc

SEQ ID NO: 25 - RNA, 74 nt, Artificial Sequence
  Synthetic Construct
ggcuagaggcggcaguccucaguacucuuacucuuuucuguaaagaggagacugcuggacucgccgcccgagcc

SEQ ID NO: 26 - RNA, 90 nt, Artificial Sequence
  Synthetic Construct
cgggucggcauggcaucuccaccuccucgcgguccgaccugggcauccgaaggaggacgcacguccacucggauggcuaagggagagcca

SEQ ID NO: 27 - RNA, 88 nt, Artificial Sequence
  Synthetic Construct
uggccggcauggucccagccuccucgcuggcgccggcugggcaacauuccgaggggaccguccccucgguaauggcgaaugggaccca

(19) United States
(12) Patent Application Publication
(10) Pub. No.: US 2024/0240218 A1
(43) Pub. Date: Jul. 18, 2024

(54) METHODS OF ENRICHING FOR CIRCULAR POLYRIBONUCLEOTIDES

(71) Applicant: Flagship Pioneering Innovations VI, LLC, Cambridge, MA (US)

(72) Inventors: Alexandra Sophie De Boer, Somerville, MA (US); Elissa Magdalene HOBERT, Cambridge, MA (US); Joseph Arthur DEPETER, Medford, MA (US)

(21) Appl. No.: 18/561,922

(22) PCT Filed: May 18, 2022
(86) PCT No.: PCT/US2022/029831

§ 371 Date: Nov. 17, 2023

Related U.S. Application Data
(60) Provisional application No. 63/190,060, filed on May 18, 2021.

(51) Int. Cl. C12P 19/34

(52) U.S. Cl. CPC C12P 19/34

(57) ABSTRACT

The methods of the disclosure can be used to enrich a population of circular polyribonucleotides in a mixture of linear polyribonucleotides and circular polyribonucleotides.

### SEQUENCE LISTING

The present application is being filed along with a Sequence Listing in electronic format. The Sequence Listing is provided as a file entitled 51509-034WO2_Sequence_Listing_5_18_22__ST25 created on May 18, 2022, which is 6,438 bytes in size. The information in electronic format of the sequence listing is incorporated herein by reference in its entirety.

### BACKGROUND OF THE INVENTION

Circular polyribonucleotides show increased resistance to degradation by nucleases resulting in a longer half-life in comparison to linear polyribonucleotides. Circular polyribonucleotides are known to occur endogenously or may be circularized exogenously. The exogenous circularization reaction results in a mixture of successfully circularized polyribonucleotides in addition to some residual linear polyribonucleotides. The presence of linear polyribonucleotides in pharmaceutical circular polyribonucleotide preparations can have unexpected and undesired effects. Thus, there remains a need for methods of enriching, separating, and/or purifying circular polyribonucleotides relative to the linear polyribonucleotides.

### SUMMARY OF THE INVENTION

This disclosure provides methods of producing an enriched population of circular polyribonucleotides. In particular, the disclosure provides methods of producing an enriched population of circular polyribonucleotides from a mixture of linear and circular polyribonucleotides by digesting the linear polyribonucleotides with a 5′ exonuclease and a 3′ exonuclease.

In one aspect, the disclosure provides a method of producing an enriched population of circular polyribonucleotides including: providing a population of polyribonucleotides including circular polyribonucleotides and linear polyribonucleotides; digesting at least a portion of the linear polyribonucleotide with a 5′ exonuclease in an amount between 0.01 U and 0.2 U (e.g., between 0.01 U and 0.16 U, 0.01 U and 0.12 U, 0.01 U and 0.0.8 U, 0.01 U and 0.04 U, 0.01 U and 0.02 U, 0.01 U and 0.012 U, 0.02 U and 0.2 U, 0.04 U and 0.2 U, 0.0.08 U and 0.2 U, 0.12 U and 0.2 U, and 0.16 U and 0.2 U) per 1 μg of polyribonucleotides; and digesting at least a portion of the linear polyribonucleotide with a 3′ exonuclease in an amount between 0.4 U and 4 U (e.g., between 0.4 U and 3.6 U, 0.4 U and 3.2 U, 0.4 U and 2.8 U, 0.4 U and 2.4 U, 0.4 U and 2 U, 0.4 U and 1.6 U, 0.4 U and 1.2 U, 0.4 U and 0.8 U, 0.8 U and 4 U, 1.2 U and 4 U, 1.6 U and 4 U, 2 U and 4 U, 24 U and 4 U, 2.8 U and 4 U, 3.2 U and 4 U, 3.6 U and 4 U, and 0.4 U and 0.6 U) per 1 μg of polyribonucleotides; thereby producing an enriched population of circular polyribonucleotides. In some embodiments, the 5′ end of at least a portion of the linear polyribonucleotides includes a monophosphate moiety. In some embodiments, the digesting step of digesting at least a portion of the linear polyribonucleotide with a 5′ exonuclease in an amount between 0.01 U and 0.2 U per 1 μg of polyribonucleotide is performed prior to digesting at least a portion of the linear polyribonucleotide with a 3′ exonuclease in an amount between 0.4 U and 4 U per 1 μg of polyribonucleotides. In some embodiments, the digesting step of digesting at least a portion of the linear polyribonucleotide with a 5′ exonuclease in an amount between 0.01 U and 0.2 U per 1 μg of polyribonucleotides is performed after digesting at least a portion of the linear polyribonucleotide with a 3′ exonuclease in an amount between 0.4 U and 4 U per 1 μg of polyribonucleotides. In some embodiments, the digesting step of digesting at least a portion of the linear polyribonucleotide with a 5′ exonuclease in an amount between 0.01 U and 0.2 U per 1 μg of polyribonucleotides and digesting at least a portion of the linear polyribonucleotide with a 3′ exonuclease in an amount between 0.4 U and 4 U per 1 μg of polyribonucleotide are performed concomitantly.

In some embodiments, the 5′ exonuclease is a 5′-phosphate dependent exonuclease. In some embodiments, the 5′ exonuclease is Xrn-1. In some embodiments, the 3′ exonuclease is RNase R. In some embodiments, the 3′ exonuclease is Exonuclease T.

In some embodiments, the digesting step of digesting at least a portion of the linear polyribonucleotide with a 5′ exonuclease in an amount between 0.01 U and 0.2 U per 1 μg of polyribonucleotides is performed for between 30 minutes and 90 minutes (e.g., between 35 minutes and 90 minutes, between 45 minutes and 90 minutes, between 1 hour and 90 minutes, between 75 minutes and 90 minutes, between 30 minutes and 75 minutes, between 30 minutes and 1 hour, between 30 minutes and 45 minutes, or between 30 minutes and 35 minutes). In some embodiments, the digesting step of digesting at least a portion of the linear polyribonucleotide with a 3′ exonuclease in an amount between 0.4 U and 4 U per 1 μg of polyribonucleotides is performed for between 30 minutes and 90 minutes (e.g., between 35 minutes and 90 minutes, between 45 minutes and 90 minutes, between 1 hour and 90 minutes, between 75 minutes and 90 minutes, between 30 minutes and 75 minutes, between 30 minutes and 1 hour, between 30 minutes and 45 minutes, or between 30 minutes and 35 minutes).

In some embodiments, the digesting step of digesting at least a portion of the linear polyribonucleotide with a 5′ exonuclease in an amount between 0.01 U and 0.2 U per 1 μg of polyribonucleotides is performed at a temperature of about 37° C. In some embodiments, the digesting step of digesting at least a portion of the linear polyribonucleotide with a 3′ exonuclease in an amount between 0.4 U and 4 U per 1 μg of polyribonucleotides is performed at a temperature of about 37° C.

In some embodiments, the digesting steps of digesting at least a portion of the linear polyribonucleotide with a 5′ exonuclease in an amount between 0.01 U and 0.2 U per 1 μg of polyribonucleotides and of digesting at least a portion of the linear polyribonucleotide with a 3′ exonuclease in an amount between 1 U and 10 U per 2.5 μg of polyribonucleotides are carried out in a digesting buffer including Mg²⁺. In some embodiments, the Mg²⁺ in the digesting buffer has a concentration between 0.05 mM and 1 mM (e.g., 0.05 mM and 0.8 mM, 0.05 mM and 0.6 mM, 0.05 mM and 0.4 mM, 0.05 mM and 0.2 mM, 0.05 mM and 0.1 mM, 0.05 mM and 0.1 mM, 0.1 mM and 1 mM, 0.2 mM and 1 mM, 0.4 mM and 1 mM, 0.6 mM and 1 mM, and 0.8 mM and 1 mM).

In some embodiments, the digesting steps of digesting at least a portion of the linear polyribonucleotide with a 5′ exonuclease in an amount between 0.01 U and 0.2 U per 1 μg of polyribonucleotides and of digesting at least a portion of the linear polyribonucleotide with a 3′ exonuclease in an amount between 0.4 U and 4 U per 1 μg of polyribonucleotides are carried out in a digesting buffer including dithiothreitol. In some embodiments, the dithiothreitol has a concentration of between 0.1 mM and 5 mM (e.g., between 0.1 mM and 4 mM, 0.1 mM and 3 mM, 0.1 mM and 2 mM, 0.1 mM and 1 mM, 0.1 mM and 0.5 mM, 0.5 mM and 5 mM, 1 mM and 5 mM, 2 mM and 5 mM, 3 mM and 5 mM, or 4 mM and 5 mM). In some embodiments, the 5′ exonuclease of the digesting step of digesting at least a portion of the linear polyribonucleotide with a 5′ exonuclease is in an amount between 0.012 U an 0.1 U (e.g., 0.012 U and 0.08 U, 0.012 U and 0.06 U, 0.012 U and 0.04 U, 0.012 U and 0.1 U, 0.02 U and 0.1 U, 0.04 U and 0.1 U, 0.06 U and 0.1 U, 0.08 U and 0.15 U) per 1 μg of polyribonucleotides. In some embodiments, the 5′ exonuclease of the digesting step of digesting at least a portion of the linear polyribonucleotide with a 5′ exonuclease is in an amount of 0.025 U per 1 μg of polyribonucleotides.

In some embodiments, the 3′ exonuclease of the digesting step of digesting at least a portion of the linear polyribonucleotide with a 3′ exonuclease is in an amount between 0.4 U and 2 U (e.g., 1 U and 1.8 U, 0.4 U and 1.6 U, 0.4 U and 1.4 U, 0.4 U and 1.2 U, 0.4 U and 1 U, 0.4 U and 0.8 U, 0.4 U and 0.6 U, 0.6 U and 2 U, 0.8 U and 2.5 U, 1 U and 2 U, 1.2 U and 2 U, 1.4 U and 2 U, 1.6 U and about 2 U, 1.8 U and 2 U) per 1 μg of polyribonucleotides. In some embodiments, the 3′ exonuclease of the digesting step of digesting at least a portion of the linear polyribonucleotide with a 3′ exonuclease is in an amount of 0.5 U per 1 μg of polyribonucleotides.

In some embodiments, following the digesting steps of digesting at least a portion of the linear polyribonucleotide with a 5′ exonuclease in an amount between 0.01 U and 0.2 U per 1 μg of polyribonucleotides and of digesting at least a portion of the linear polyribonucleotide with a 3′ exonuclease in an amount between 0.4 U and 4 U per 1 μg of polyribonucleotides, the percent (w/w) of the circular polyribonucleotides is between 40% and 95% (e.g., between 40% and 90%, 40% and 80%, 40% and 70%, 40% and 60%, 40% and 50%, 50% and 95%, 60% and 95%, 70% and 95%, 80% and 95%, or 90% and 95%) of the total polynucleotides. In some embodiments, following the digesting steps of digesting at least a portion of the linear polyribonucleotide with a 5′ exonuclease in an amount between 0.01 U and 0.2 U per 1 μg of polyribonucleotides and of digesting at least a portion of the linear polyribonucleotide with a 3′ exonuclease in an amount between 0.4 U and 4 U per 1 μg of polyribonucleotides, the percent (w/w) of the circular polyribonucleotides is between 60% and 90% (e.g., between 60% and 85%, 60% and 80%, 60% and 75%, 60% and 70%, 60% and 65%, 65% and 90%, 70% and 90%, 75% and 90%, 80% and 90%, or 85% and 90%) of the total polynucleotides. In some embodiments, following the digesting steps of digesting at least a portion of the linear polyribonucleotide with a 5′ exonuclease in an amount between 0.01 U and 0.2 U per 1 μg of polyribonucleotides and of digesting at least a portion of the linear polyribonucleotide with a 3′ exonuclease in an amount between 0.4 U and 4 U per 1 μg of polyribonucleotides, the percent (w/w) of the circular polyribonucleotides is between 70% and 90% (e.g., between 70% and 90%, 70% and 85%, 70% and 80%, 70% and 75%, 75% and 90%, 80% and 90%, or 85% and 90%) of the total nucleotides. In some embodiments, following the digesting steps of digesting at least a portion of the linear polyribonucleotide with a 5′ exonuclease in an amount between 0.01 U and 0.2 U per 1 μg of polyribonucleotides and of digesting at least a portion of the linear polyribonucleotide with a 3′ exonuclease in an amount between 0.4 U and 4 U per 1 μg of polyribonucleotides, the overall percent yield (w/w) of circular polyribonucleotide is between 70% and 100% (e.g., between 70% and 95%, 70% and 90%, 70% and 85%, 70% and 80%, 70% and 75%, 75% and 100%, 80% and 100%, 85% and 100%, 90% and 100%, and 95% and 100%).

In another aspect, the disclosure provides a method of producing an enriched population of circular polyribonucleotides including: providing a population of polyribonucleotides including circular polyribonucleotides and linear polyribonucleotides; digesting at least a portion of the linear polyribonucleotide with a 5′ exonuclease, and digesting at least a portion of the linear polyribonucleotide with a 3′ exonuclease, wherein the digesting steps of digesting at least a portion of the linear polyribonucleotide with a 5′ exonuclease and digesting at least a portion of the linear polyribonucleotide with a 3′ exonuclease are performed in a digesting buffer including between 0.05 mM and 1 mM (e.g., 0.05 mM and 0.8 mM, 0.05 mM and 0.6 mM, 0.05 mM and 0.4 mM, 0.05 mM and 0.2 mM, 0.05 mM and 0.1 mM, 0.05 mM and 0.1 mM, 0.1 mM and 1 mM, 0.2 mM and 1 mM, 0.4 mM and 1 mM, 0.6 mM and 1 mM, and 0.8 mM and 1 mM) Mg²⁺ and/or between 0.1 mM and 5 mM (e.g., between 0.1 mM and 4 mM, 0.1 mM and 3 mM, 0.1 mM and 2 mM, 0.1 mM and 1 mM, 0.1 mM and 0.5 mM, 0.5 mM and 5 mM, 1 mM and 5 mM, 2 mM and 5 mM, 3 mM and 5 mM, or 4 mM and 5 mM) dithiothreitol; hereby producing an enriched population of circular polyribonucleotides.

In some embodiments, the digesting step of digesting at least a portion of the linear polyribonucleotide with a 5′ exonuclease is performed prior to the step of digesting at least a portion of the linear polyribonucleotide with a 3′ exonuclease. In some embodiments, the digesting step of digesting at least a portion of the linear polyribonucleotide with a 5′ exonuclease is performed after the step of digesting at least a portion of the linear polyribonucleotide with a 3′ exonuclease. In some embodiments, the digesting steps of digesting at least a portion of the linear polyribonucleotide with a 5′ exonuclease and of digesting at least a portion of the linear polyribonucleotide with a 3′ exonuclease are performed concomitantly.

In some embodiments, the 5′ exonuclease in an amount between 0.01 U and 0.2 U (e.g., between 0.01 U and 0.16 U, 0.01 U and 0.12 U, 0.01 U and 0.0.8 U, 0.01 U and 0.04 U, 0.01 U and 0.02 U, 0.01 U and 0.012 U, 0.02 U and 0.2 U, 0.04 U and 0.2 U, 0.0.08 U and 0.2 U, 0.12 U and 0.2 U, and 0.16 U and 0.2 U) per 1 μg of polyribonucleotides. In some embodiments, the 3′ exonuclease in an amount between 0.4 U and 4 U (e.g., between 0.4 U and 3.6 U, 0.4 U and 3.2 U, 0.4 U and 2.8 U, 0.4 U and 2.4 U, 0.4 U and 2 U, 0.4 U and 1.6 U, 0.4 U and 1.2 U, 0.4 U and 0.8 U, 0.8 U and 4 U, 1.2 U and 4 U, 1.6 U and 4 U, 2 U and 4 U, 24 U and 4 U, 2.8 U and 4 U, 3.2 U and 4 U, 3.6 U and 4 U, and 0.4 U and 0.6 U) per 1 μg of polyribonucleotides.

In embodiments, the 5′ end of at least a portion of the linear polyribonucleotides includes a monophosphate moiety.

In some embodiments, the 5′ exonuclease is a 5′-phosphate dependent exonuclease. In some embodiments, the 5′ exonuclease is Xrn-1. In some embodiments, the 3′ exonuclease is RNase R. In some embodiments, the 3′ exonuclease is Exonuclease T.

In some embodiments, the digesting step of digesting at least a portion of the linear polyribonucleotide with a 5′ exonuclease is performed for between 30 minutes and 90 minutes (e.g., between 35 minutes and 90 minutes, between 45 minutes and 90 minutes, between 1 hour and 90 minutes, between 75 minutes and 90 minutes, between 30 minutes and 75 minutes, between 30 minutes and 1 hour, between 30 minutes and 45 minutes, or between 30 minutes and 35 minutes). In some embodiments, the digesting step of digesting at least a portion of the linear polyribonucleotide with a 5′ exonuclease is performed for between 45 minutes and 60 minutes (e.g., between 45 minutes and 55 minutes, between 45 minutes and 50 minutes, between 50 minutes and 60 minutes, and between 55 minutes and 60 minutes). In some embodiments, the digesting step of digesting at least a portion of the linear polyribonucleotide with a 3′ exonuclease is performed for between 30 minutes and 90 minutes (e.g., between 35 minutes and 90 minutes, between 45 minutes and 90 minutes, between 1 hour and 90 minutes, between 75 minutes and 90 minutes, between 30 minutes and 75 minutes, between 30 minutes and 1 hour, between 30 minutes and 45 minutes, or between 30 minutes and 35 minutes). In some embodiments, the digesting step of digesting at least a portion of the linear polyribonucleotide with a 3′ exonuclease is performed for between 45 minutes and 60 minutes (e.g., between 45 minutes and 55 minutes, between 45 minutes and 50 minutes, between 50 minutes and 60 minutes, and between 55 minutes and 60 minutes).

In some embodiments, the digesting step of digesting at least a portion of the linear polyribonucleotide with a 5′ exonuclease is performed at a temperature of about 37° C. In some embodiments, the digesting step of digesting at least a portion of the linear polyribonucleotide with a 3′ exonuclease is performed at a temperature of about 37° C.

In some embodiments, the 5′ exonuclease of the digesting step of digesting at least a portion of the linear polyribonucleotide with a 5′ exonuclease is in an amount between 0.012 U an 0.1 U (e.g., 0.012 U and 0.08 U, 0.012 U and 0.06 U, 0.012 U and 0.04 U, 0.012 U and 0.02 U, 0.02 U and 0.1 U, 0.04 U and 0.1 U, 0.06 U and 0.1 U, 0.08 U and 0.1 U) per 1 μg of polyribonucleotides. In some embodiments, the 5′ exonuclease of the digesting step of digesting at least a portion of the linear polyribonucleotide with a 5′ exonuclease is in an amount of 0.025 U per 1 μg of polyribonucleotides.

In some embodiments, the 3′ exonuclease of the digesting step of digesting at least a portion of the linear polyribonucleotide with a 3′ exonuclease is in an amount between 0.4 U and 2 U (e.g., 0.4 U and 1.8 U, 0.4 U and 1.6 U, 0.4 U and 1.4 U, 0.4 U and 1.2 U, 0.4 U and 1 U, 0.4 U and 0.8 U, 0.4 U and 0.6 U, 0.6 U and 2 U, 0.8 U and 2 U, 1 and 2 U, 1.2 U and 2 U, 1.4 U and 2 U, 1.6 U and 2 U, 1.8 U and 2 U) per 1 μg of polyribonucleotides. In some embodiments, the 3′ exonuclease of digesting step of digesting at least a portion of the linear polyribonucleotide with a 3′ exonuclease is in an amount of 0.5 U per 1 μg of polyribonucleotides.

In some embodiments, following the digesting steps of digesting at least a portion of the linear polyribonucleotide with a 5′ exonuclease and digesting at least a portion of the linear polyribonucleotide with a 3′ exonuclease, the percent (w/w) of the circular polyribonucleotides is between 40% and 95% (e.g., between 40% and 90%, 40% and 80%, 40% and 70%, 40% and 60%, 40% and 50%, 50% and 95%, 60% and 95%, 70% and 95%, 80% and 95%, or 90% and 95%) of the total polynucleotides.

In some embodiments, following the digesting steps of digesting at least a portion of the linear polyribonucleotide with a 5′ exonuclease and digesting at least a portion of the linear polyribonucleotide with a 5′ exonuclease, the percent (w/w) of the circular polyribonucleotides is between 60% and 90% (e.g., between 60% and 85%, 60% and 80%, 60% and 75%, 60% and 70%, 60% and 65%, 65% and 90%, 70% and 90%, 75% and 90%, 80% and 90%, or 85% and 90%) of the total polynucleotides. In some embodiments, following the digesting steps of digesting at least a portion of the linear polyribonucleotide with a 5′ exonuclease and digesting at least a portion of the linear polyribonucleotide with a 5′ exonuclease, the percent (w/w) of the circular polyribonucleotides is between 70% and 90% (e.g., between 70% and 90%, 70% and 85%, 70% and 80%, 70% and 75%, 75% and 90%, 80% and 90%, or 85% and 90%) of the total nucleotides. In some embodiments, following the digesting steps of digesting at least a portion of the linear polyribonucleotide with a 5′ exonuclease and digesting at least a portion of the linear polyribonucleotide with a 3′ exonuclease the overall percent yield (w/w) of circular polyribonucleotide is between 70% and 100%.

### Definitions

To facilitate the understanding of this invention, a number of terms are defined below. Terms defined herein have meanings as commonly understood by a person of ordinary skill in the areas relevant to the invention. Terms such as “a,” “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, any values provided in a range of values include both the upper and lower bounds, and any values contained within the upper and lower bounds.

As used herein, the term “3′ exonuclease” refers to an enzyme having exonuclease activity wherein the enzyme removes nucleotides from a strand of nucleotides using hydrolysis starting from the 3′ end of the strand of nucleotides and continuing in a processive manner toward the 5′ end of the strand of nucleotides. 3′ exonucleases include but are not limited to Exonuclease T, RNase PH, Polynucleotide Phosphorylase, RNase D, RNase R, and Exoribonuclease II.

As used herein, the term “5′ exonuclease” refers to an enzyme having exonuclease activity wherein the enzyme removes nucleotides from a strand of nucleotides using hydrolysis starting from the 5′ end of the strand of nucleotides and continuing in a processive manner toward the 3′ end of the strand of nucleotides. 5′ exonucleases include but are not limited to Xrn-1, and Terminator™ exonuclease.

As used herein, the term “5′-phosphate dependent exonuclease” refers to an exonuclease that digests polynucleotides having a 5′ monophosphate in a 5′-to-3′ processive manner (e.g., Terminator™ exonuclease and Xrn-1).

As used herein, the terms “circRNA,” “circular polyribonucleotide,” “circular RNA,” and “circular polyribonucleotide molecule” are used interchangeably and mean a polyribonucleotide molecule that has a structure having no free ends (i.e., no free 3′ or 5′ end), for example a polyribonucleotide molecule that forms a circular or end-less structure through covalent or non-covalent bonds.

As used herein, the term “circularization efficiency” is a measurement of resultant circular polyribonucleotide versus its non-circular starting material.

As used herein, the terms “circRNA preparation,” “circular polyribonucleotide preparation,” and “circular RNA preparation” are used interchangeably and mean a composition including circRNA molecules and a diluent, carrier, first adjuvant, or a combination thereof.

As used herein, the term “digested mixture” refers to a mixture including linear polyribonucleotides and circular polyribonucleotides that is produced by contacting a mixture of linear polyribonucleotides and circular polyribonucleotides with a digesting enzyme (e.g., a 5′ exonuclease or 3′ exonuclease).

As used herein, the terms “digesting buffer” and “digestion buffer” refer to a buffer in which a nuclease (e.g., an exonuclease) is active and digests at least a portion of a polynucleotide. A digesting buffer may include components such as a buffering agent, Mg²⁺, and/or dithiothreitol (DTT).

As used herein, the term “enriched population” refers to a population polyribonucleotides that has a higher percentage of circular polyribonucleotides in comparison to another population of circular and linear polyribonucleotides.

As used herein, the terms “fragment” and “portion” mean any part of a polynucleotide molecule that is at least one nucleotide shorter than the polynucleotide molecule. For example, a nucleotide molecule can be a linear polyribonucleotide molecule and a fragment thereof can be a monoribonucleotide or any number of contiguous polyribonucleotides that are a portion of the linear polyribonucleotide molecule.

As used herein, the term “impurity” is an undesired substance present in a composition, e.g., a pharmaceutical composition as described herein. In some embodiments, an impurity is a process-related impurity. In some embodiments, an impurity is a product-related substance other than the desired product in the final composition, e.g., other than the active drug ingredient, e.g., circular, or linear polyribonucleotide, as described herein. As used herein, the term “process-related impurity” is a substance used, present, or generated in the manufacturing of a composition, preparation, or product that is undesired in the final composition, preparation, or product other than the linear polyribonucleotides described herein. In some embodiments, the process-related impurity is an enzyme used in the synthesis or circularization of polyribonucleotides. As used herein, the term “product-related substance” is a substance or byproduct produced during the synthesis of a composition, preparation, or product, or any intermediate thereof. In some embodiments, the product-related substance is deoxyribonucleotide fragments. In some embodiments, the product-related substance is deoxyribonucleotide monomers. In some embodiments, the product-related substance is one or more of: derivatives or fragments of polyribonucleotides described herein, e.g., fragments of 10, 9, 8, 7, 6, 5, or 4 ribonucleic acids, monoribonucleic acids, diribonucleic acids, or triribonucleic acids.

As used herein, the term “linear counterpart” refers to a polyribonucleotide molecule (and its fragments) having the same or similar nucleotide sequence (e.g., 100%, 95%, 90%, 85%, 80%, 75%, or any percentage therebetween sequence identity) as a circular polyribonucleotide and having two free ends (i.e., the uncircularized version (and its fragments) of the circularized polyribonucleotide). In some embodiments, the linear counterpart (e.g., a pre-circularized version) is a polyribonucleotide molecule (and its fragments) having the same or similar nucleotide sequence (e.g., 100%, 95%, 90%, 85%, 80%, 75%, or any percentage therebetween sequence identity) and same or similar nucleic acid modifications as a circular polyribonucleotide and having two free ends (i.e., the uncircularized version (and its fragments) of the circularized polyribonucleotide). In some embodiments, the linear counterpart is a polyribonucleotide molecule (and its fragments) having the same or similar nucleotide sequence (e.g., 100%, 95%, 90%, 85%, 80%, 75%, or any percentage therebetween sequence identity) and different or no nucleic acid modifications as a circular polyribonucleotide and having two free ends (i.e., the uncircularized version (and its fragments) of the circularized polyribonucleotide). In some embodiments, a fragment of the polyribonucleotide molecule that is the linear counterpart is any portion of linear counterpart polyribonucleotide molecule that is shorter than the linear counterpart polyribonucleotide molecule. In some embodiments, the linear counterpart further includes a 5′ cap. In some embodiments, the linear counterpart further includes a poly adenosine tail. In some embodiments, the linear counterpart further includes a 3′ UTR. In some embodiments, the linear counterpart further includes a 5′ UTR.

As used herein, the terms “linear RNA,” “linear polyribonucleotide,” and “linear polyribonucleotide molecule” are used interchangeably and mean polyribonucleotide molecule having a 5′ and 3′ end. One or both of the 5′ and 3′ ends may be free ends or joined to another moiety. Linear RNA includes RNA that has not undergone circularization (e.g., is pre-circularized) and can be used as a starting material for circularization through, for example, splint ligation, or chemical, enzymatic, ribozyme- or splicing-catalyzed circularization methods.

As used herein, the term “mixture” means a material made of two or more different substances that are mixed. In some cases, a mixture described herein can be a homogenous mixture of the two or more different substances, e.g., the mixture can have the same proportions of its components (e.g., the two or more substances) throughout any given sample of the mixture. In some cases, a mixture as provided herein can be a heterogeneous mixture of the two or more different substances, e.g., the proportions of the components of the mixture (e.g., the two or more substances) can vary throughout the mixture. In some cases, the mixture includes circular polyribonucleotides and linear polyribonucleotides. In some embodiments, the mixture includes circular polyribonucleotides, linear polyribonucleotides, and may include linear polydeoxyribonucleotides. In some cases, a mixture is a liquid solution, e.g., the mixture is present in liquid phase. In some instances, a liquid solution can be regarded as comprising a liquid solvent and a solute. Mixing a solute in a liquid solvent can be termed as “dissolution” process. In some cases, a liquid solution is a liquid-in-liquid solution (e.g., a liquid solute dissolved in a liquid solvent), a solid-in-liquid solution (e.g., a solid solute dissolved in a liquid solvent), or a gas-in-liquid solution (e.g., a solid solute dissolved in a liquid solvent). In some cases, there is more than one solvent and/or more than one solute. In some cases, a mixture is a colloid, liquid suspension, or emulsion. In some cases, a mixture is a solid mixture, e.g., the mixture is present in solid phase.

As used herein, the term “modified ribonucleotide” means a nucleotide with at least one modification to the sugar, the nucleobase, or the internucleoside linkage.

The term “polynucleotide” as used herein means a molecule comprising one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose.

Polydeoxyribonucleotides or deoxyribonucleic acids, or DNA, means macromolecules that include multiple deoxyribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a polynucleotide is deoxyribonucleic acid (DNA), ribonucleic acid (RNA), or derivatives or variants thereof. In some cases, a polynucleotide is a short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), to name a few, and encompasses both the nucleotide sequence and any structural embodiments thereof, such as single-stranded, double-stranded, triple-stranded, helical, hairpin, etc. In some cases, a polynucleotide molecule is circular. A polynucleotide can have various lengths. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. As embodied herein, the polynucleotide sequences may include isolated and purified DNA/RNA molecules, synthetic DNA/RNA molecules, and synthetic DNA/RNA analogs.

Polynucleotides, e.g., polyribonucleotides or polydeoxyribonucleotides, may include one or more nucleotide variants, including nonstandard nucleotide(s), non-natural nucleotide(s), nucleotide analog(s) and/or modified nucleotides. Modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as amino ally 1-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A. Nat. Chem. Biol. 2012 July; 8(7):612-4, which is herein incorporated by reference for all purposes.

As used herein, the phrase “quasi-helical structure” is a higher order structure of the circular polyribonucleotide, wherein at least a portion of the circular polyribonucleotide folds into a helical structure.

As used herein, the terms “total ribonucleotide molecules” and “total polyribonucleotides” mean the total amount of any ribonucleotide molecules, including linear polyribonucleotide molecules, circular polyribonucleotide molecules, monomeric ribonucleotides, other polyribonucleotide molecules, fragments thereof, and modified variations thereof, as measured by total mass of the ribonucleotide molecules. As used herein, the term “unit” refers to the amount of enzyme required to perform a defined catalytic activity under specified methods of the assay method which are summarized for each enzyme in Table 1.

TABLE 1
Definitions of a unit for various enzymes

| Enzyme | One unit (U) is equal to the amount of enzyme that: |
|---|---|
| Xrn-1 | digests 1 μg of phosphorylated yeast RNA in 60 |
| | minutes at 37° C. in 100 mM NaCl, 50 mM Tris-HCl, 10 |
| | mM MgCl₂ and 1 mM dithiothreitol with a pH of 7.9 at |
| | 25° C. |
| RNase R | converts 1 μg of poly-r(A) into acid-soluble nucleotides |
| | in 10 minutes at 37° C. in 20 mM Tris-HCl (pH 8.0), 100 |
| | mM KCl and 0.1 mM MgCl₂. |
| Terminator ™ | digests 1 μg of rRNA substrate into acid-soluble |
| | nucleotides in 60 minutes at 30° C. under standard |
| | assay conditions. |
| Exonuclease T | produces 0.1 nmol of trichloroacetic acetic acid (TCA) |
| | soluble DNA from 1 nmol of [3H]-labeled polythymidine |
| | in 30 minutes at 25° C. in a total reaction volume of 100 |
| | μl in 50 mM potassium acetate, 20 mM Tris-acetate, 10 |
| | mM magnesium acetate, and 1 mM dithiothreitol with |
| | pH of 7.9 at 25° C. |
| Polynucleotide | polymerizes 1.0 μmol of ADP, releasing 1.0 μmol of |
| phosphorylase | inorganic phosphate in 15 minutes, at pH 9.1 at 37° C. |
| | in a solution of 20 mM HEPES buffer pH 7.9, 0.1 mM |
| | EDTA, 2 mM DTT, 12.5 mM MgCl2, ~130 mM KCl, |
| | and 20% (w/v) glycerol. |
| RNase PH | degrades 0.07 nmol of tRNA-CCA-[³H]C₂₋₃ after 30 |
| | minutes at 37° C. in 50 mM Tris-Cl, pH 8.0, 50 mM |
| | KCl, 5 mM MgCl₂, and 10 mM NaPO₄ as described in |
| | Kelly and Deutscher, J. Biol. Chem. 1992 Aug. |
| | 25; 267(24): 17153-8. |

### BRIEF DESCRIPTION OF THE DRAWINGS

FIG. 1 shows that linear polyribonucleotide encoding gLuc is degraded after digestion with RNase R.

FIG. 2 shows that linear polyribonucleotide encoding gLuc is degraded after digestion with Terminator™ exonuclease.

FIG. 3 shows that linear polyribonucleotide encoding gLuc is degraded after digestion with Xrn-1.

FIG. 4 shows that a combination of Terminator™ exonuclease and RNase R selectively degrades linear polyribonucleotide encoding gLuc.

FIG. 5 shows enrichment of circular polyribonucleotide in the post-ligation polyribonucleotide mixture using a combination of Terminator™ exonuclease and RNase R.

FIG. 6 shows that digestion concomitantly with 5′ and 3′ exonucleases in optimized concentrations selectively degraded linear polyribonucleotides in the post-ligation polyribonucleotide mixture, producing an enriched circular polyribonucleotide preparation.

FIG. 7 shows enrichment of circular polyribonucleotide using two different combinations of 5′ and 3′ exonucleases.

FIG. 8 shows the results of the digestions of a post-ligation polyribonucleotide mixture with the combination of Terminator™ exonuclease and RNase R in various buffers and corresponding buffer controls.

FIG. 9 shows the results of the digestions of a post-ligation polyribonucleotide mixture with the combination of Xrn-1 and RNase R in various buffers and corresponding buffer controls.

FIG. 10 shows the results of the digestions of a post-ligation polyribonucleotide mixture with the combination of Xrn-1 and RNase R in various buffers and corresponding buffer controls.

FIG. 11 shows in vitro expression of Gaussia Luciferase from enriched circular polyribonucleotide.

FIG. 12 shows the results of selective enrichment of circular polyribonucleotides of several different molecular weights.

FIG. 13 shows the results of the concomitant digestions using several different combinations of 5′ and 3′ exonucleases.

### DETAILED DESCRIPTION

The present disclosure provides methods for producing an enriched a population of circular polyribonucleotides. For example, using the compositions and methods described herein an enriched population of circular polyribonucleotides may be produced by providing a population of polyribonucleotides including circular polyribonucleotides and linear polyribonucleotides; digesting at least a portion of the linear polyribonucleotide with a 5′ exonuclease in an amount between 0.01 U and 0.2 U per 1 μg of polyribonucleotides; and digesting at least a portion of the linear polyribonucleotide with a 3′ exonuclease in an amount between 0.4 U and 4 U per 1 μg of polyribonucleotides. The present disclosure also provides methods of producing an enriched population of circular polyribonucleotides by providing a population of polyribonucleotides including circular polyribonucleotides and linear polyribonucleotides; digesting at least a portion of the linear polyribonucleotide with a 5′ exonuclease; and digesting at least a portion of the linear polyribonucleotide with a 3′ exonuclease, wherein the digesting steps are performed in a digesting buffer comprising between 0.05 mM and 1 mM Mg²⁺, thereby producing an enriched population of circular polyribonucleotides.

The population of polyribonucleotides including circular and linear polyribonucleotides may be reacted with the 5′ exonuclease and 3′ exonuclease at a concentration, for a time, and at a temperature that is sufficient to allow for at least a portion of the linear polyribonucleotides to be digested.

### Methods of Enriching for Circular Polyribonucleotide

In one aspect the disclosure provides a method of producing an enriched population of circular polyribonucleotides. The method of enriching a population polyribonucleotides for circular polyribonucleotides may include providing a population of polyribonucleotides comprising circular polyribonucleotides and linear polyribonucleotides; digesting at least a portion of the linear polyribonucleotide with a 5′ exonuclease; digesting at least a portion of the linear polyribonucleotide with a 3′ exonuclease, thereby producing an enriched population of circular polyribonucleotides. The 5′ exonuclease may be in an amount of between 0.01 U and 0.25 U per 1 μg of polyribonucleotides, and the 3′ exonuclease may be in an amount between 0.4 U and 4 U per 1 μg of polyribonucleotides.

In another aspect, the disclosure provides a method of producing an enriched population of circular polyribonucleotides providing a population of polyribonucleotides comprising circular polyribonucleotides and linear polyribonucleotides; digesting at least a portion of the linear polyribonucleotide with a 5′ exonuclease; digesting at least a portion of the linear polyribonucleotide with a 3′ exonuclease, wherein the digesting buffer includes Mg²⁺ and/or dithiothreitol (DTT); thereby producing an enriched population of circular polyribonucleotides.

### Exonuclease Digestion

In an aspect, the population of polyribonucleotides including circular polyribonucleotides and linear polyribonucleotides is reacted with a 5′ exonuclease and a 3′ exonuclease to produce an enriched population of circular polyribonucleotides.

In some embodiments, at least a portion of the linear polyribonucleotides is digested with a 5′ exonuclease. In some embodiments, at least a portion of the linear polyribonucleotides is digested with the 3′ exonuclease. In some embodiments, at least a portion of the linear polyribonucleotides is digested with the 5′ exonuclease prior to digesting with the 3′ exonuclease. In some embodiments, at least a portion of the linear polyribonucleotides is digested with the 3′ exonuclease prior to digesting with the 5′ exonuclease. In some embodiments, at least a portion of the linear polyribonucleotides is digested with the 5′ exonuclease and the 3′ exonuclease concomitantly.

In some embodiments, the 5′ exonuclease is a 5′-phosphate dependent exonuclease (e.g., Xrn-1 or Terminator™ exonuclease). In some embodiments, the 5′ exonuclease is Xrn-1. In some embodiments, the 5′ exonuclease is Terminator™ exonuclease. In some embodiments, the 3′ exonuclease is RNase R. In some embodiments, the 3′ exonuclease is Exonuclease T. In some embodiments, the 3′ exonuclease is RNase PH. In some embodiments, the 3′ exonuclease is Polynucleotide Phosphorylase. In some embodiments, the 3′ exonuclease is RNase D. In some embodiments, the 3′ exonuclease is Exoribonuclease II.

In some embodiments, the 5′ exonuclease is in amount of between 0.01 U and 0.2 U (e.g., between 0.01 U and 0.16 U, 0.01 U and 0.12 U, 0.01 U and 0.0.8 U, 0.01 U and 0.04 U, 0.01 U and 0.02 U, 0.01 U and 0.012 U, 0.02 U and 0.2 U, 0.04 U and 0.2 U, 0.0.08 U and 0.2 U, 0.12 U and 0.2 U, and 0.16 U and 0.2 U) per 1 μg of polyribonucleotides. In some embodiments, the 5′ exonuclease is in an amount between 0.012 U an 0.1 U (e.g., 0.012 U and 0.08 U, 0.012 U and 0.06 U, 0.012 U and 0.04 U, 0.012 U and 0.02 U, 0.02 U and 0.1 U, 0.04 U and 0.1 U, 0.06 U and 0.1 U, 0.08 U and 0.1 U) per 1 μg of polyribonucleotides. In some embodiments, the 5′ exonuclease is in an amount of 0.025 U per 1 μg of polyribonucleotides.

In some embodiments, the 3′ exonuclease is in amount of between 0.4 U and 4 U (e.g., between 0.4 U and 3.6 U, 0.4 U and 3.2 U, 0.4 U and 2.8 U, 0.4 U and 2.4 U, 0.4 U and 2 U, 0.4 U and 1.6 U, 0.4 U and 1.2 U, 0.4 U and 0.8 U, 0.8 U and 4 U, 1.2 U and 4 U, 1.6 U and 4 U, 2 U and 4 U, 24 U and 4 U, 2.8 U and 4 U, 3.2 U and 4 U, 3.6 U and 4 U, and 0.4 U and 0.6 U) per 1 μg of polyribonucleotides. In some embodiments, the 3′ exonuclease is in an amount between 0.4 U and 2 U (e.g., 0.4 U and 1.8 U, 0.4 U and 1.6 U, 0.4 U and 1.4 U, 0.4 U and 1.2 U, 0.4 U and 1 U, 0.4 U and 0.8 U, 0.4 U and 0.6 U, 0.6 U and 2 U, 0.8 U and 2 U, 1 U and 2 U, 1.2 U and 2 U, 1.4 U and 2 U, 1.6 U and 2 U, 1.8 U and 2 U) per 1 μg of polyribonucleotides. In some embodiments, the 3′ exonuclease is in an amount of 0.5 U per 1 μg of polyribonucleotides.

In some embodiments, the ratio of the concentration of 5′ exonuclease to the concentration of 3′ exonuclease is 0.2 U 5′ exonuclease to 4 U 3′ exonuclease per 1 μg of polyribonucleotides.

In some embodiments, the reaction of the 5′ exonuclease with the population of circular polyribonucleotides including linear polyribonucleotides and circular polyribonucleotides is performed for at least 30 minutes (e.g., at least 35 minutes, at least 1 hour, at least 1.5 hours, at least 2 hours, at least 5 hours, at least 12 hours, or at least 24 hours). In some embodiments, the reaction of the 5′ exonuclease with the population of circular polyribonucleotides including linear polyribonucleotides and circular polyribonucleotides is performed for between 30 minutes and 90 minutes (e.g., between 35 minutes and 90 minutes, between 45 minutes and 90 minutes, between 1 hour and 90 minutes, between 30 minutes and 75 minutes, between 30 minutes and 1 hour, between 30 minutes and 45 minutes, or between 30 minutes and 35 minutes). In some embodiments, the digesting step of digesting at least a portion of the linear polyribonucleotide with a 5′ exonuclease is performed for between 45 minutes and 60 minutes (e.g., between 45 minutes and 55 minutes, between 45 minutes and 50 minutes, between 50 minutes and 60 minutes, and between 55 minutes and 60 minutes).

In some embodiments, the reaction of the 3′ exonuclease with the population of circular polyribonucleotides including linear polyribonucleotides and circular polyribonucleotides is performed for at least 30 minutes (e.g., at least 35 minutes, at least 1 hour, at least 1.5 hours, at least 2 hours, at least 5 hours, at least 12 hours, or at least 24 hours). In some embodiments, the reaction of the 5′ exonuclease with the population of circular polyribonucleotides including linear polyribonucleotides and circular polyribonucleotides is performed for between 30 minutes and 90 minutes (e.g., between 35 minutes and 90 minutes, between 45 minutes and 90 minutes, between 1 hour and 90 minutes, between 30 minutes and 75 minutes, between 30 minutes and 1 hour, between 30 minutes and 45 minutes, or between 30 minutes and 35 minutes). In some embodiments, the digesting step of digesting at least a portion of the linear polyribonucleotide with a 3′ exonuclease is performed for between 45 minutes and 60 minutes (e.g., between 45 minutes and 55 minutes, between 45 minutes and 50 minutes, between 50 minutes and 60 minutes, and between 55 minutes and 60 minutes).

In some embodiments, the reaction of the 5′ exonuclease with the population of circular polyribonucleotides including linear polyribonucleotides and circular polyribonucleotides is performed at a temperature of between 30° C. and 42° C. (e.g., about 31° C., 32° C., 33° C., 34° C., 35° C., 36° C., 37° C., 38° C., 39° C., 40° C., and 41° C.). In some embodiments, the reaction of the 5′ exonuclease with the population of circular polyribonucleotides including linear polyribonucleotides and circular polyribonucleotides is performed at a temperature of about 37° C. In some embodiments, the reaction of the 3′ exonuclease with the population of circular polyribonucleotides including linear polyribonucleotides and circular polyribonucleotides is performed at a temperature of between 30° C. and 42° C. (e.g., about 31° C., 32° C., 33° C., 34° C., 35° C., 36° C., 37° C., 38° C., 39° C., 40° C., and 41° C.). In some embodiments, the reaction of the 3′ exonuclease with the population of circular polyribonucleotides including linear polyribonucleotides and circular polyribonucleotides is performed at a temperature of about 37° C.

Digesting Buffer In an aspect, the population of polyribonucleotides including circular polyribonucleotides and linear polyribonucleotides is reacted with a 5′ exonuclease and a 3′ exonuclease to produce an enriched population of circular polyribonucleotides in a digesting buffer.

In some embodiments, the digesting buffer includes between 0.05 mM and 1 mM (e.g., 0.05 mM and 0.8 mM, 0.05 mM and 0.6 mM, 0.05 mM and 0.4 mM, 0.05 mM and 0.2 mM, 0.05 mM and 0.1 mM, 0.05 mM and 0.1 mM, 0.1 mM and 1 mM, 0.2 mM and 1 mM, 0.4 mM and 1 mM, 0.6 mM and 1 mM, and 0.8 mM and 1 mM) Mg²⁺. In some embodiments, the digesting buffer includes between 0.05 mM and 0.5 mM (e.g., 0.05 mM and 0.1 mM, 0.05 mM and 0.2 mM, 0.05 mM and 0.3 mM, 0.05 mM and 0.4 mM, 0.1 mM and 0.5 mM, 0.2 mM and 0.5 mM, 0.3 mM and 0.5 mM, and 0.4 mM and 0.5 mM) Mg²⁺. In some embodiments, the digesting buffer includes about 0.1 mM Mg²⁺. In some embodiments, the digesting buffer includes about 0.5 mM Mg²⁺. The Mg²⁺ may be added to the digesting buffer as magnesium sulfate, magnesium chloride, magnesium lactate, magnesium citrate, magnesium carbonate, or any magnesium salt.

In some embodiments, the digesting buffer includes dithiothreitol (DTT). In some embodiments, the DTT has a concentration of between 0.1 mM and 5 mM (e.g., between 0.1 mM and 4 mM, 0.1 mM and 3 mM, 0.1 mM and 2 mM, 0.1 mM and 1 mM, 0.1 mM and 0.5 mM, 0.5 mM and 5 mM, 1 mM and 5 mM, 2 mM and 5 mM, 3 mM and 5 mM, or 4 mM and 5 mM) in the digesting buffer. In some embodiments, the DTT has a concentration of between 0.5 mM and 2 mM (e.g., between 0.5 mM and 0.7 mM, 0.5 mM and 1 mM, 0.5 mM and 1.2 mM, 0.5 mM and 1.5 mM, 0.5 mM and 1.7 mM, 1.7 mM and 2 mM, 1.5 mM and 2 mM, 1.2 mM and 2 mM, 1 mM and 2 mM, or 0.8 mM and 2 mM) in the digesting buffer. In some embodiments, the DTT has a concentration of 1 mM in the digesting buffer.

In some embodiments, the digesting buffer includes NaCl. In some embodiments, the digesting buffer includes between 10 mM and 1 M NaCl; for example, the digesting buffer may include between 10 mM and 900 mM, 10 mM and 700 mM, 10 mM and 500 mM, 10 mM and 200 mM, 10 mM and 100 mM, 10 mM and 50 mM, 50 mM and 1 M, 100 mM and 1 M, 200 mM and 1 M, 500 mM and 1 M, or 700 mM and 1 M NaCl. In some embodiments, the digesting buffer includes between 10 mM and 200 mM NaCl. In some embodiments, the digesting buffer includes 100 mM NaCl.

In some embodiments, the digesting buffer includes a buffering agent. In some embodiments, the buffering agent comprises Tris-HCl, Tris base, HEPES, phosphate buffered saline (PBS), or a combination thereof. For example, the buffering agent may be Tris-HCl. In some embodiments, the digesting buffer includes 10 mM and 1 M of the buffering agent; for example, the digesting buffer may include between 10 mM and 900 mM, 10 mM and 700 mM, 10 mM and 500 mM, 10 mM and 200 mM, 10 mM and 100 mM, 10 mM and 50 mM, 50 mM and 1 M, 100 mM and 1 M, 200 mM and 1 M, 500 mM and 1 M, or 700 mM and 1 M of the buffering agent. In some embodiments, the digesting buffer includes 10 mM and 500 mM of the buffering agent; for example, the digesting buffer may include between 10 mM and 40 mM, 10 mM and 400 mM, 10 mM and 350 mM, 10 mM and 300 mM, 10 mM and 250 mM, 10 mM and 200 mM, 10 mM and 150 mM, 10 mM and 100 mM, 10 mM and 50 mM, 50 mM and 500 mM, 100 mM and 500 mM, 150 mM and 500 mM, 200 mM and 500 mM, 250 mM and 500 mM, 300 mM and 500 mM, 350 mM and 500 mM, 400 mM and 500 mM, and 450 mM and 500 mM. In some embodiments, the digesting buffer includes 50 mM of the buffering agent.

In some embodiments, the digesting buffer has a pH between 6 and 8. For example, the pH of the digesting buffer may be between 6 and 7.8, 6 and 7.5, 6 and 7.2, 6 and 7, 6 and 6.8, 6 and 6.5, 6 and 6.3, 6.3 and 8, 6.5 and 8, 6.8 and 8, 7 and 8, 7.3 and 8, 7.5 and 8, and 7.8 and 8.

### Enriched Population of Circular Polyribonucleotides

In some embodiments, following digestion with the 5′ exonuclease and the 3′ exonuclease the percent (w/w) of the circular polyribonucleotides is between 40% and 95% (e.g., between 40% and 90%, 40% and 80%, 40% and 70%, 40% and 60%, 40% and 50%, 50% and 95%, 60% and 95%, 70% and 95%, 80% and 95%, or 90% and 95%) of the total polynucleotides. In some embodiments, following digestion with the 5′ exonuclease and the 3′ exonuclease the percent (w/w) of the circular polyribonucleotides is between 60% and 90% (e.g., between 60% and 85%, 60% and 80%, 60% and 75%, 60% and 70%, 60% and 65%, 65% and 90%, 70% and 90%, 75% and 90%, 80% and 90%, or 85% and 90%) of the total polynucleotides. In some embodiments, following digestion with the 5′ exonuclease and the 3′ exonuclease the percent (w/w) of the circular polyribonucleotides is between 70% and 90% (e.g., between 70% and 90%, 70% and 85%, 70% and 80%, 70% and 75%, 75% and 90%, 80% and 90%, or 85% and 90%) of the total polynucleotides. In some embodiments, following digestion with the 5′ exonuclease and the 3′ exonuclease the overall percent yield (w/w) of circular polyribonucleotide is between 60% and 100%; for example, the overall percent yield (w/w) of the circular polyribonucleotides is between 60% and 95%, 60% and 90%, 60% and 85%, 60% and 80%, 60% and 75%, 60% and 70%, 60% and 65%, 65% and 100%, 70% and 100%, 75% and 100%, 80% and 100%, 85% and 100%, 90% and 100%, and 95% and 100%. In some embodiments, the overall percent yield (w/w) of the circular polyribonucleotide is between 70% and 100%; for example, the percent yield (w/w) of the circular polyribonucleotides is between 70% and 95%, 70% and 90%, 70% and 85%, 70% and 80%, 70% and 75%, 75% and 100%, 80% and 100%, 85% and 100%, 90% and 100%, and 95% and 100%. In some embodiments, the overall percent yield (w/w) of the circular polyribonucleotide is between 80% and 100%; for example, the overall percent yield (w/w) of the circular polyribonucleotide is between 80% and 95%, 80% and 90%, 80% and 85%, 85% and 100%, 90% and 100%, and 95% and 100%.

### Circular Polyribonucleotides

The present disclosure provides a population of circular polyribonucleotides that may be enriched from a mixture of circular polyribonucleotides and linear polyribonucleotides.

In some embodiments, the circular polyribonucleotide includes one or more of the elements as described herein. In some embodiments, the circular polyribonucleotide lacks a poly-A sequence (e.g., lacks a polyA sequence at the 3′ end of the ORF), lacks a free 3′ end, lacks an RNA polymerase recognition motif, or any combination thereof. In some embodiments, the circular polyribonucleotide includes any feature, or any combination of features as disclosed in International Patent Publication No. WO2019/118919, which is hereby incorporated by reference in its entirety.

In some embodiments, the polyribonucleotide (e.g., the circular polyribonucleotide) is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the polyribonucleotide (e.g., the circular polyribonucleotide) is less than 1.2 kb, is 1.2-1.4 kb, is 1.4-1.6 kb, is 3.5-4.5 kb, is 4.5-5.4 kb, or is greater than 5.4 kb

In some embodiments, the polyribonucleotide (e.g., the circular polyribonucleotide) may be of a sufficient size to accommodate a binding site for a ribosome. In some embodiments, the maximum size of a circular polyribonucleotide can be as large as is within the technical constraints of producing a circular polyribonucleotide, and/or using the circular polyribonucleotide. Without wishing to be bound by any particular theory, it is possible that multiple segments of RNA may be produced from DNA and their 5′ and 3′ free ends annealed to produce a “string” of RNA, which ultimately may be circularized when only one 5′ and one 3′ free end remains. In some embodiments, the maximum size of a circular polyribonucleotide may be limited by the ability of packaging and delivering the RNA to a target. In some embodiments, the size of a circular polyribonucleotide is a length sufficient to encode useful polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 400 nucleotides, at least 300 nucleotides, at least 200 nucleotides, at least 100 nucleotides, or at least 70 nucleotides, may be useful.

In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent, e.g., that is comparable to or similar to in the absence of exonuclease. In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein. In some embodiments, the circular polyribonucleotide lacks a 5′ cap.

### Expression Sequences

In some embodiments, the circular polyribonucleotide includes an expression sequence that encodes a peptide or polypeptide. In some embodiments, the circular polyribonucleotide includes at least one expression sequence that encodes a peptide or polypeptide. Such peptide may include, but is not limited to, small peptide, peptidomimetic (e.g., peptoid), amino acids, and amino acid analogs. The peptide may be linear or branched. Such peptide may have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. Such peptide may include, but is not limited to, a neurotransmitter, a hormone, a drug, a toxin, a viral or microbial particle, a synthetic molecule, and agonists or antagonists thereof.

The encoded polypeptide may have a length from about 5 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less may be useful.

The polypeptide may be produced in substantial amounts. As such, the polypeptide may be any proteinaceous molecule that can be produced. A polypeptide can be a polypeptide that can be secreted from a cell, or localized to the cytoplasm, nucleus, or membrane compartment of a cell. Some polypeptides include, but are not limited to, at least a portion of a viral envelope protein, metabolic regulatory enzymes (e.g., that regulate lipid or steroid production), an antigen, a cytokine, a toxin, enzymes whose absence is associated with a disease, and polypeptides that are not active in an animal until cleaved (e.g., in the gut of an animal), and a hormone.

In some embodiments, the circular polyribonucleotide includes an expression sequence encoding a protein e.g., a therapeutic protein. In some embodiments, therapeutic proteins that can be expressed from the circular polyribonucleotide disclosed herein have antioxidant activity, binding, cargo receptor activity, catalytic activity, molecular carrier activity, molecular function regulator, molecular transducer activity, nutrient reservoir activity, protein tag, structural molecule activity, toxin activity, transcription regulator activity, translation regulator activity, or transporter activity. Some examples of therapeutic proteins may include, but are not limited to, an enzyme replacement protein, a protein for supplementation, a protein vaccination, antigens (e.g. tumor antigens, viral, bacterial), hormones, cytokines, antibodies, immunotherapy (e.g. cancer), cellular reprogramming/transdifferentiation factor, transcription factors, chimeric antigen receptor, transposase or nuclease, immune effector (e.g., influences susceptibility to an immune response/signal), a regulated death effector protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, and a CRISPR system or component thereof.

In some embodiments, exemplary proteins that can be expressed from the circular polyribonucleotide disclosed herein include human proteins, for instance, receptor binding protein, hormone, growth factor, growth factor receptor modulator, and regenerative protein (e.g., proteins implicated in proliferation and differentiation, e.g., therapeutic protein, for wound healing). In some embodiments, exemplary proteins that can be expressed from the circular polyribonucleotide disclosed herein include EGF (epithelial growth factor). In some embodiments, exemplary proteins that can be expressed from the circular polyribonucleotide disclosed herein include enzymes, for instance, oxidoreductase enzymes, metabolic enzymes, mitochondrial enzymes, oxygenases, dehydrogenases, ATP-independent enzyme, and desaturases. In some embodiments, exemplary proteins that can be expressed from the circular polyribonucleotide disclosed herein include an intracellular protein or cytosolic protein. In some embodiments, the circular polyribonucleotide expresses a NanoLuc® luciferase (nLuc). In some embodiments, exemplary proteins that can be expressed from the circular polyribonucleotide disclosed herein include a secretary protein, for instance, a secretary enzyme. In some cases, the circular polyribonucleotide expresses a secretary protein that can have a short half-life therapeutic in the blood, or can be a protein with a subcellular localization signal, or protein with secretory signal peptide. In some embodiments, the circular polyribonucleotide expresses a Gaussia Luciferase (gLuc). In some cases, the circular polyribonucleotide expresses a non-human protein, for instance, a fluorescent protein, an energy-transfer acceptor, or a protein-tag like Flag, Myc, or His. In some embodiments, exemplary proteins that can be expressed from the circular polyribonucleotide include a GFP. In some embodiments, the circular polyribonucleotide expresses tagged proteins, .e.g., fusion proteins or engineered proteins containing a protein tag, e.g., chitin binding protein (CBP), maltose binding protein (MBP), Fc tag, glutathione-S-transferase (GST), AviTag, Calmodulin-tag, polyglutamate tag; E-tag, FLAG-tag), HA-tag, His-tag, Myc-tag, NE-tag, S-tag, SBP-tag, Softag 1, Softag 3, Spot-tag, Strep-tag; TC tag, Ty tag, V5 tag; VSV-tag; or Xpress tag.

In some embodiments, the circular polyribonucleotide encodes the expression of an antibody, e.g., an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can comprise more than one expression sequence, each of which expresses a portion of an antibody, and the sum of which can constitute the antibody. In some cases, the circular polyribonucleotide comprises one expression sequence coding for the heavy chain of an antibody, and another expression sequence coding for the light chain of the antibody. In some cases, when the circular polyribonucleotide is expressed in a cell or a cell-free environment, the light chain and heavy chain can be subject to appropriate modification, folding, or other post-translation modification to form a functional antibody.

### Regulatory Elements

In some embodiments, a circular polyribonucleotide includes a regulatory element, e.g., a sequence that modifies expression of an expression sequence within the circular polyribonucleotide. A regulatory element may include a sequence that is located adjacent to an expression sequence that encodes an expression product. A regulatory element may be operably linked to the adjacent sequence. A regulatory element may increase an amount of product expressed as compared to an amount of the expressed product when no regulatory element is present. A regulatory element may be used to increase the expression of one or more polypeptides encoded by a circular polyribonucleotide. Likewise, a regulatory element may be used to decrease the expression of one or more polypeptides encoded by a circular polyribonucleotide. In some embodiments, a regulatory element may be used to increase expression of a polypeptide and another regulatory element may be used to decrease expression of another polypeptide on the same circular polyribonucleotide. In addition, one regulatory element can increase an amount of a product (e.g., a polypeptide) expressed for multiple expression sequences attached in tandem. Hence, one regulatory element can enhance the expression of one or more expression sequences (e.g., polypeptides). Multiple regulatory elements can also be used, for example, to differentially regulate expression of different expression sequences. In some embodiments, a regulatory element as provided herein can include a selective translation sequence. As used herein, the term “selective translation sequence” refers to a nucleic acid sequence that selectively initiates or activates translation of an expression sequence in the circular polyribonucleotide, for instance, certain riboswitch aptazymes. A regulatory element can also include a selective degradation sequence. As used herein, the term “selective degradation sequence” refers to a nucleic acid sequence that initiates degradation of the circular polyribonucleotide, or an expression product of the circular polyribonucleotide. In some embodiments, the regulatory element is a translation modulator. A translation modulator can modulate translation of the expression sequence in the circular polyribonucleotide. A translation modulator can be a translation enhancer or suppressor. In some embodiments, a translation initiation sequence can function as a regulatory element. Further examples of regulatory elements are described in paragraphs [0154]-[0161] of International Patent Publication No. WO2019/118919, which is hereby incorporated by reference in its entirety.

Nucleotides flanking a codon that initiates translation, such as, but not limited to, a start codon or an alternative start codon, are known to affect the translation efficiency, the length and/or the structure of the circular polyribonucleotide. (See e.g., Matsuda and Mauro PLoS ONE, 2010 5: 11; the contents of which are herein incorporated by reference in its entirety). Masking any of the nucleotides flanking a codon that initiates translation may be used to alter the position of translation initiation, translation efficiency, length and/or structure of the circular polyribonucleotide.

In one embodiment, a masking agent may be used near the start codon or alternative start codon in order to mask or hide the codon to reduce the probability of translation initiation at the masked start codon or alternative start codon. In another embodiment, a masking agent may be used to mask a start codon of the circular polyribonucleotide in order to increase the likelihood that translation will initiate at an alternative start codon.

### Translation Initiation Sequences

In some embodiments, a circular polyribonucleotide encodes a polypeptide and includes a translation initiation sequence, e.g., a start codon. In some embodiments, the translation initiation sequence includes a Kozak or Shine-Dalgarno sequence. In some embodiments, the translation initiation sequence includes a Kozak sequence. In some embodiments, the circular polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the circular polyribonucleotide includes at least one translation initiation sequence adjacent to an expression sequence. In some embodiments, the translation initiation sequence provides conformational flexibility to the circular polyribonucleotide. In some embodiments, the translation initiation sequence is within a substantially single stranded region of the circular polyribonucleotide. Further examples of translation initiation sequences are described in paragraphs [0163]-[0165] of International Patent Publication No. WO2019/118919, which is hereby incorporated by reference in its entirety.

The circular polyribonucleotide may include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation may initiate on the first start codon or may initiate downstream of the first start codon.

In some embodiments, a circular polyribonucleotide may initiate at a codon which is not the first start codon, e.g., AUG. Translation of the circular polyribonucleotide may initiate at an alternative translation initiation sequence, such as those described in [0164] of International Patent Publication No. WO2019/118919, which is incorporated herein by reference in its entirety.

In some embodiments, translation is initiated by eukaryotic initiation factor 4A (eIF4A) treatment with Rocaglates (translation is repressed by blocking 43S scanning, leading to premature, upstream translation initiation and reduced protein expression from transcripts bearing the RocA-eIF4A target sequence, see for example, www.nature.com/articles/nature17978).

### Stagger Elements

A circular polyribonucleotide of the disclosure can include a cleavage domain (e.g., a stagger element or a cleavage sequence). The term “stagger element” refers to a moiety, such as a nucleotide sequence, that induces ribosomal pausing during translation. In some embodiments, the stagger element is a non-conserved sequence of amino-acids with a strong alpha-helical propensity followed by the consensus sequence—D(V/I)ExNPGP—(SEQ ID NO: 2), where x=any amino acid. In some embodiments, the stagger element may include a chemical moiety, such as glycerol, a non-nucleic acid linking moiety, a chemical modification, a modified nucleic acid, or any combination thereof.

In some embodiments, a circular polyribonucleotide includes at least one stagger element adjacent to an expression sequence. In some embodiments, the stagger element is present on one or both sides of each expression sequence, leading to separation of the expression products. In some embodiments, the stagger element is a portion of the one or more expression sequences. In some embodiments, the circular polyribonucleotide includes one or more expression sequences, and each of the one or more expression sequences is separated from a succeeding expression sequence. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element includes a portion of an expression sequence of the one or more expression sequences.

Examples of stagger elements are described in paragraphs [0172]-[0175] of International Patent Publication No. WO2019/118919, which is hereby incorporated by reference in its entirety.

To avoid production of a continuous expression product while maintaining rolling circle translation, a stagger element may be included to induce ribosomal pausing during translation. In some embodiments, the stagger element is at the 3′ end of at least one of the one or more expression sequences. The stagger element can be configured to stall a ribosome during rolling circle translation of the circular polyribonucleotide. The stagger element may include, but is not limited to a 2A-like, or CHYSEL (cis-acting hydrolase element) sequence. In some embodiments, the stagger element encodes a sequence with a C-terminal consensus sequence that is X₁X₂X₃EX₅NPGP (SEQ ID NO: 22), where X₁ is absent or G or H, X₂ is absent or D or G, X₃ is D or V or I or S or M, and X₅ is any amino acid. In some embodiments, this sequence includes a non-conserved sequence of amino-acids with a strong alpha-helical propensity followed by the consensus sequence −D(V/I)ExNPGP (SEQ ID NO: 2), where x=any amino acid. Some non-limiting examples of stagger elements includes GDVESNPGP (SEQ ID NO: 3), GDIEENPGP (SEQ ID NO: 4), VEPNPGP (SEQ ID NO: 5), IETNPGP (SEQ ID NO: 6), GDIESNPGP (SEQ ID NO: 7), GDVELNPGP (SEQ ID NO: 8), GDIETNPGP (SEQ ID NO: 9), GDVENPGP (SEQ ID NO: 10), GDVEENPGP (SEQ ID NO: 11), GDVEQNPGP (SEQ ID NO: 12), IESNPGP (SEQ ID NO: 13), GDIELNPGP (SEQ ID NO: 14), HDIETNPGP (SEQ ID NO: 15), HDVETNPGP (SEQ ID NO: 16), HDVEMNPGP (SEQ ID NO: 17), GDMESNPGP (SEQ ID NO: 18), GDVETNPGP (SEQ ID NO: 19), GDIEQNPGP ((SEQ ID NO: 20), and DSEFNPGP (SEQ ID NO: 21).

In some embodiments, a stagger element described herein cleaves an expression product, such as between G and P of the consensus sequence described herein. As one non-limiting example, the circular polyribonucleotide includes at least one stagger element to cleave the expression product. In some embodiments, the circular polyribonucleotide includes a stagger element adjacent to at least one expression sequence. In some embodiments, the circular polyribonucleotide includes a stagger element after each expression sequence. In some embodiments, the circular polyribonucleotide includes a stagger element which is present on one or both sides of each expression sequence, leading to translation of individual peptide(s) and or polypeptide(s) from each expression sequence.

In some embodiments, a stagger element includes one or more modified nucleotides or unnatural nucleotides that induce ribosomal pausing during translation. Unnatural nucleotides may include peptide nucleic acid (PNA), Morpholino and locked nucleic acid (LNA), as well as glycol nucleic acid (GNA) and threose nucleic acid (TNA). Examples such as these are distinguished from naturally occurring DNA or RNA by changes to the backbone of the molecule. Modifications can include any modification to the sugar, the nucleobase, the internucleoside linkage (e.g., to a linking phosphate/to a phosphodiester linkage/to the phosphodiester backbone), and any combination thereof that can induce ribosomal pausing during translation. Some of the exemplary modifications provided herein are described elsewhere herein.

In some embodiments, a stagger element is present in a circular polyribonucleotide in other forms. For example, in some exemplary circular polyribonucleotides, a stagger element includes a termination element of a first expression sequence in the circular polyribonucleotide, and a nucleotide spacer sequence that separates the termination element from a first translation initiation sequence of an expression succeeding the first expression sequence. In some examples, the first stagger element of the first expression sequence is upstream of (5′ to) a first translation initiation sequence of the expression succeeding the first expression sequence in the circular polyribonucleotide. In some cases, the first expression sequence and the expression sequence succeeding the first expression sequence are two separate expression sequences in the circular polyribonucleotide. The distance between the first stagger element and the first translation initiation sequence can enable continuous translation of the first expression sequence and its succeeding expression sequence. In some embodiments, the first stagger element includes a termination element and separates an expression product of the first expression sequence from an expression product of its succeeding expression sequences, thereby creating discrete expression products. In some cases, the circular polyribonucleotide including the first stagger element upstream of the first translation initiation sequence of the succeeding sequence in the circular polyribonucleotide is continuously translated, while a corresponding circular polyribonucleotide including a stagger element of a second expression sequence that is upstream of a second translation initiation sequence of an expression sequence succeeding the second expression sequence is not continuously translated. In some cases, there is only one expression sequence in the circular polyribonucleotide, and the first expression sequence and its succeeding expression sequence are the same expression sequence. In some exemplary circular polyribonucleotides, a stagger element includes a first termination element of a first expression sequence in the circular polyribonucleotide, and a nucleotide spacer sequence that separates the termination element from a downstream translation initiation sequence. In some such examples, the first stagger element is upstream of (5′ to) a first translation initiation sequence of the first expression sequence in the circular polyribonucleotide. In some cases, the distance between the first stagger element and the first translation initiation sequence enables continuous translation of the first expression sequence and any succeeding expression sequences. In some embodiments, the first stagger element separates one round expression product of the first expression sequence from the next round expression product of the first expression sequences, thereby creating discrete expression products. In some cases, the circular polyribonucleotide including the first stagger element upstream of the first translation initiation sequence of the first expression sequence in the circular polyribonucleotide is continuously translated, while a corresponding circular polyribonucleotide including a stagger element upstream of a second translation initiation sequence of a second expression sequence in the corresponding circular polyribonucleotide is not continuously translated. In some cases, the distance between the second stagger element and the second translation initiation sequence is at least 2×, 3×, 4×, 5×, 6×, 7×, 8×, 9×, or 10× greater in the corresponding circular polyribonucleotide than a distance between the first stagger element and the first translation initiation in the circular polyribonucleotide. In some cases, the distance between the first stagger element and the first translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater. In some embodiments, the distance between the second stagger element and the second translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater than the distance between the first stagger element and the first translation initiation. In some embodiments, the circular polyribonucleotide includes more than one expression sequence.

### IRES

In some embodiments, a circular polyribonucleotide described herein includes an internal ribosome entry site (IRES) element. In some embodiments, a circular polyribonucleotide described herein includes more than one (e.g., 2, 3, 4, and 5) internal ribosome entry site (IRES) element. In some embodiments, the circular polyribonucleotide includes one or more IRES sequences on one or both sides of each expression sequence, leading to separation of the resulting peptide(s) and or polypeptide(s). In some embodiments, the IRES flanks both sides of at least one (e.g., 2, 3, 4, 5 or more) expression sequence. A suitable IRES element to include in a circular polyribonucleotide can be an RNA sequence capable of engaging a eukaryotic ribosome. In some embodiments, the IRES is an encephalomyocarditis virus (EMCV) IRES. In some embodiments, the IRES is a Coxsackievirus (CVB3) IRES. Further examples of an IRES are described in paragraphs [0166]-[0168] of International Patent Publication No. WO2019/118919, which is hereby incorporated by reference in its entirety. In some embodiments, the circular polyribonucleotide lacks an internal ribosomal entry site. In some embodiments, the circular polyribonucleotide lacks an internal ribosomal entry site and is competent for protein expression from its one or more expression sequences.

### Translation

In some embodiments, once translation of a circular polyribonucleotide is initiated, the ribosome bound to the circular polyribonucleotide does not disengage from the circular polyribonucleotide before finishing at least one round of translation of the circular polyribonucleotide. In some embodiments, the circular polyribonucleotide as described herein is competent for rolling circle translation. In some embodiments, during rolling circle translation, once translation of the circular polyribonucleotide is initiated, the ribosome bound to the circular polyribonucleotide does not disengage from the circular polyribonucleotide before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 105 rounds, or at least 106 rounds of translation of the circular polyribonucleotide.

In some embodiments, the rolling circle translation of a circular polyribonucleotide leads to generation of polypeptide product that is translated from more than one round of translation of the circular polyribonucleotide (“continuous” expression product). In some embodiments, the circular polyribonucleotide includes a stagger element, and rolling circle translation of the circular polyribonucleotide leads to generation of polypeptide product that is generated from a single round of translation or less than a single round of translation of the circular polyribonucleotide (“discrete” expression product). In some embodiments, the circular polyribonucleotide is configured such that at least 10%, 20%, 30%, 40%, 50%, at least 60%, at least 70%, at least 80%, at least 90%, at least 95%, at least 96%, at least 97%, at least 98%, at least 99%, or 100% of total polypeptides (molar/molar) generated during the rolling circle translation of the circular polyribonucleotide are discrete polypeptides. In some embodiments, the circular polyribonucleotide is configured such that at least 99% of the total polypeptides are discrete polypeptides. In some embodiments, the amount ratio of the discrete products over the total polypeptides is tested in an in vitro translation system. In some embodiments, the in vitro translation system used for the test of amount ratio includes rabbit reticulocyte lysate. In some embodiments, the amount ratio is tested in an in vivo translation system, such as a eukaryotic cell or a prokaryotic cell, a cultured cell, or a cell in an organism.

### Untranslated Regions

In some embodiments, a circular polyribonucleotide includes untranslated regions (UTRs). UTRs of a genomic region including a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for a first expression sequence is the same as or continuous with or overlapping with another UTR for a second expression sequence. In some embodiments, the intron is a human intron. In some embodiments, the intron is a full-length human intron, e.g., ZKSCAN1.

In some embodiments, a circular polyribonucleotide includes a UTR with one or more stretches of adenosines and uridines embedded within. These AU rich signatures may increase turnover rates of the expression product.

Introduction, removal, or modification of UTR AU rich elements (AREs) may be useful to modulate the stability, or immunogenicity (e.g., the level of one or more marker of an immune or inflammatory response) of the circular polyribonucleotide. When engineering specific circular polyribonucleotides, one or more copies of an ARE may be introduced to the circular polyribonucleotide and the copies of an ARE may modulate translation and/or production of an expression product. Likewise, AREs may be identified and removed or engineered into the circular polyribonucleotide to modulate the intracellular stability and thus affect translation and production of the resultant protein.

It should be understood that any UTR from any gene may be incorporated into the respective flanking regions of the circular polyribonucleotide. Exemplary untranslated regions are described in paragraphs [0197]-[201] of International Patent Publication No. WO2019/118919, which is hereby incorporated by reference in its entirety.

In some embodiments, a circular polyribonucleotide lacks a 5′-UTR and is competent for protein expression from its one or more expression sequences. In some embodiments, the circular polyribonucleotide lacks a 3′-UTR and is competent for protein expression from its one or more expression sequences. In some embodiments, a circular polyribonucleotide lacks a 5′-UTR. In some embodiments, the circular polyribonucleotide lacks a 3′-UTR. In some embodiments, the circular polyribonucleotide lacks a termination element and is competent for protein expression from its one or more expression sequences. In some embodiments, the circular polyribonucleotide lacks a cap and is competent for protein expression from its one or more expression sequences. In some embodiments, the circular polyribonucleotide lacks a 5′-UTR, a 3′-UTR, and an IRES, and is competent for protein expression from its one or more expression sequences. In some embodiments, the circular polyribonucleotide includes one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, IncRNAs, shRNA), and a sequence that encodes a therapeutic mRNA or protein.

### Termination Elements

A circular polyribonucleotide can include one or more expression sequences and each expression sequence may or may not have a termination element. Further examples of termination elements are described in paragraphs [0169]-[0170] of International Patent Publication No. WO2019/118919, which is hereby incorporated by reference in its entirety.

In some embodiments, a circular polyribonucleotide includes a poly-A sequence. In some embodiments, the length of a poly-A sequence is greater than 10 nucleotides in length. In one embodiment, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A sequence is designed according to the descriptions of the poly-A sequence in [0202]-[0204] of International Patent Publication No. WO2019/118919, which is incorporated herein by reference in its entirety. In some embodiments, a circular polyribonucleotide lacks a poly-A sequence (e.g., lacks a poly-A sequence at the 3′ end of the ORF). In some embodiments, the circular polyribonucleotide lacks a termination element. In some embodiments, the circular polyribonucleotide lacks a poly-A sequence (e.g., lacks a polyA sequence at the 3′ end of the ORF) and is competent for protein expression from its one or more expression sequences

In some embodiments, the circular polyribonucleotide includes one or more expression sequences and the expression sequences lack a termination element, such that the circular polyribonucleotide is continuously translated. Exclusion of a termination element may result in rolling circle translation or continuous expression of expression product, e.g., peptides or polypeptides, due to lack of ribosome stalling or fall-off. In such an embodiment, rolling circle translation expresses a continuous expression product through each expression sequence. In some other embodiments, a termination element of an expression sequence can be part of a stagger element. In some embodiments, one or more expression sequences in the circular polyribonucleotide comprises a termination element. However, rolling circle translation or expression of a succeeding (e.g., second, third, fourth, fifth, etc.) expression sequence in the circular polyribonucleotide is performed. In such instances, the expression product may fall off the ribosome when the ribosome encounters the termination element, e.g., a stop codon, and terminates translation. In some embodiments, translation is terminated while the ribosome, e.g., at least one subunit of the ribosome, remains in contact with the circular polyribonucleotide.

In some embodiments, the circular polyribonucleotide includes a termination element at the end of one or more expression sequences. In some embodiments, one or more expression sequences comprises two or more termination elements in succession. In such embodiments, translation is terminated and rolling circle translation is terminated. In some embodiments, the ribosome completely disengages with the circular polyribonucleotide. In some such embodiments, production of a succeeding (e.g., second, third, fourth, fifth, etc.) expression sequence in the circular polyribonucleotide may require the ribosome to reengage with the circular polyribonucleotide prior to initiation of translation. Generally, termination elements include an in-frame nucleotide triplet that signals termination of translation, e.g., UAA, UGA, UAG. In some embodiments, one or more termination elements in the circular polyribonucleotide are frame-shifted termination elements, such as but not limited to, off-frame or −1 and +1 shifted reading frames (e.g., hidden stop) that may terminate translation. Frame-shifted termination elements include nucleotide triples, TAA, TAG, and TGA that appear in the second and third reading frames of an expression sequence. Frame-shifted termination elements may be important in preventing misreads of mRNA, which is often detrimental to the cell.

### RNA Binding Sites

In some embodiments, the circular polyribonucleotide comprises one or more target RNA binding sites. In some embodiments, the circular polyribonucleotide includes target RNA binding sites that modify expression of an endogenous gene and/or an exogenous gene. In some embodiments, the target RNA binding site modulates expression of a host gene. The target RNA binding site can include a sequence that hybridizes to an endogenous gene (e.g., a sequence for a miRNA, siRNA, mRNA, lncRNA, RNA, DNA, an antisense RNA, gRNA as described herein), a sequence that hybridizes to an exogenous nucleic acid such as a viral DNA or RNA, a sequence that hybridizes to an RNA, a sequence that interferes with gene transcription, a sequence that interferes with RNA translation, a sequence that stabilizes RNA or destabilizes RNA such as through targeting for degradation, or a sequence that modulates a DNA- or RNA-binding factor. In some embodiments, the circular polyribonucleotide comprises a target aptamer sequence that binds to an RNA. The target aptamer sequence can bind to an endogenous gene (e.g., a sequence for a miRNA, siRNA, mRNA, lncRNA, RNA, DNA, an antisense RNA, gRNA as described herein), to an exogenous nucleic acid such as a viral DNA or RNA, to an RNA, to a sequence that interferes with gene transcription, to a sequence that interferes with RNA translation, to a sequence that stabilizes RNA or destabilizes RNA such as through targeting for degradation, or to a sequence that modulates a DNA- or RNA-binding factor. The secondary structure of the target aptamer sequence can bind to the RNA. The circular RNA can form a complex with the RNA by binding of the target aptamer sequence to the RNA.

In some embodiments, the target RNA binding site can be one of a tRNA, lncRNA, lincRNA, miRNA, rRNA, snRNA, microRNA, siRNA, piRNA, snoRNA, snRNA, exRNA, scaRNA, Y RNA, and hnRNA binding site. Target RNA binding sites are well-known to persons of ordinary skill in the art.

Certain target RNA binding sites can inhibit gene expression through the biological process of RNA interference (RNAi). In some embodiments, the circular polyribonucleotides comprises an RNAi molecule with RNA or RNA-like structures typically having 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNA (siRNA), double-strand RNA (dsRNA), microRNA (miRNA), short hairpin RNA (shRNA), meroduplexes, and dicer substrates. Further examples of target binding sites are described in paragraphs [0129]-[0146] of WO2020/023655, which is hereby incorporated by reference in its entirety

### DNA Binding Sites

In some embodiments, the circular polyribonucleotide comprises a target DNA binding site, such as a sequence for a guide RNA (gRNA). In some embodiments, the circular polyribonucleotide comprises a guide RNA or a complement to a gRNA sequence. A gRNA short synthetic RNA composed of a target binding sequence necessary for binding to the incomplete effector moiety and a user-defined ˜20 nucleotide targeting sequence for a genomic target. Guide RNA sequences can have a length of between 17-24 nucleotides (e.g., 19, 20, or 21 nucleotides) and complementary to the targeted nucleic acid sequence. Custom gRNA generators and algorithms can be used in the design of effective guide RNA. Gene editing can be achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNA can be effective in genome editing.

The gRNA can recognize specific DNA sequences (e.g., sequences adjacent to or within a promoter, enhancer, silencer, or repressor of a gene).

In some embodiments, the gRNA is part of a CRISPR system for gene editing. For gene editing, the circular polyribonucleotide can be designed to include one or multiple guide RNA sequences corresponding to a desired target DNA sequence. The gRNA sequences may include at least 10, 11, 12, 13, 14, 15, 16, 17, 18, 19, 20, 21, 22, 23, 24, 25, 26, 27, 28, 29, 30 or more nucleotides for interaction with Cas9 or other exonuclease to cleave DNA, e.g., Cpf1 interacts with at least about 16 nucleotides of gRNA sequence for detectable DNA cleavage.

In some embodiments, the circular polyribonucleotide comprises a target aptamer sequence that can bind to DNA. The secondary structure of the target aptamer sequence can bind to DNA. In some embodiments, the circular polyribonucleotide forms a complex with the DNA by binding of the target aptamer sequence to the DNA.

Further examples of circular polyribonucleotide sequences that bind to DNA are described in paragraphs [0151]-[0153] of WO2020/023655, which is hereby incorporated by reference in its entirety.

### Protein-Binding

In some embodiments, the circular polyribonucleotide includes one or more protein binding sites that enable a protein, e.g., a ribosome, to bind to an internal site in the RNA sequence. By engineering protein binding sites, e.g., ribosome binding sites, into the circular polyribonucleotide, the circular polyribonucleotide may evade or have reduced detection by the host's immune system, have modulated degradation, or modulated translation, by masking the circular polyribonucleotide from components of the host's immune system.

In some embodiments, the circular polyribonucleotide comprises at least one immunoprotein binding site, for example to evade immune responses, e.g., CTL (cytotoxic T lymphocyte) responses. In some embodiments, the immunoprotein binding site is a nucleotide sequence that binds to an immunoprotein and aids in masking the circular polyribonucleotide as exogenous. In some embodiments, the immunoprotein binding site is a nucleotide sequence that binds to an immunoprotein and aids in hiding the circular polyribonucleotide as exogenous or foreign.

Traditional mechanisms of ribosome engagement to linear RNA involve ribosome binding to the capped 5′ end of an RNA. From the 5′ end, the ribosome migrates to an initiation codon, whereupon the first peptide bond is formed. According to the present invention, internal initiation (i.e., cap-independent) of translation of the circular polyribonucleotide does not require a free end or a capped end. Rather, a ribosome binds to a non-capped internal site, whereby the ribosome begins polypeptide elongation at an initiation codon. In some embodiments, the circular polyribonucleotide includes one or more RNA sequences comprising a ribosome binding site, e.g., an initiation codon.

Natural 5′UTRs bear features which play roles in for translation initiation. They harbor signatures like Kozak sequences which are commonly known to be involved in the process by which the ribosome initiates translation of many genes. Kozak sequences have the consensus CCR(A/G)CCAUGG (SEQ ID NO: 23), where R is a purine (adenine or guanine) three bases upstream of the start codon (AUG), which is followed by another ‘G’. 5′UTR also have been known to form secondary structures which are involved in elongation factor binding.

In some embodiments, the circular polyribonucleotide encodes a protein binding sequence that binds to a protein. In some embodiments, the protein binding sequence targets or localizes the circular polyribonucleotide to a specific target. In some embodiments, the protein binding sequence specifically binds an arginine-rich region of a protein.

In some embodiments, the protein binding site includes, but is not limited to, a binding site to the protein such as ACIN1, AGO, APOBEC3F, APOBEC3G, ATXN2, AUH, BCCIP, CAPRINI, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIP1 L1, FKBP4, FMR1, FUS, FXR1, FXR2, GNL3, GTF2F1, HNRNPA1, HNRNPA2B1, HNRNPC, HNRNPK, HNRNPL, HNRNPM, HNRNPU, HNRNPUL1, IGF2BP1, IGF2BP2, IGF2BP3, ILF3, KHDRBS1, LARP7, LIN28A, LIN28B, m6A, MBNL2, METTL3, MOV10, MSI1, MSI2, NONO, NONO—, NOP58, NPM1, NUDT21, PCBP2, POLR2A, PRPF8, PTBP1, RBFOX2, RBM10, RBM22, RBM27, RBM47, RNPS1, SAFB2, SBDS, SF3A3, SF3B4, SIRT7, SLBP, SLTM, SMNDC1, SND1, SRRM4, SRSF1, SRSF3, SRSF7, SRSF9, TAF15, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, AKT1, and any other protein that binds RNA.

### Modifications

A circular polyribonucleotide described herein may include one or more substitutions, insertions and/or additions, deletions, and covalent modifications with respect to reference sequences, in particular, the parent polyribonucleotide, are included within the scope of this invention.

In some embodiments, a circular polyribonucleotide includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nuc/Acids Res 27: 196-197). In some embodiments, the first isolated nucleic acid includes messenger RNA (mRNA). In some embodiments, the mRNA includes at least one nucleoside selected from the group such as those described in [0311] of International Patent Publication No. WO2019/118919, which is incorporated herein by reference in its entirety.

A circular polyribonucleotide may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, a circular polyribonucleotide includes at least one N(6)methyladenosine (m6A) modification to increase translation efficiency. In some embodiments, the N(6)methyladenosine (m6A) modification can reduce immunogenicity (e.g., reduce the level of one or more marker of an immune or inflammatory response) of the circular polyribonucleotide.

In some embodiments, a modification may include a chemical or cellular induced modification. For example, some non-limiting examples of intracellular RNA modifications are described by Lewis and Pan in “RNA modifications and structures cooperate to guide RNA-protein interactions” from Nat Reviews Mol Cell Biol, 2017, 18:202-210.

In some embodiments, chemical modifications to the ribonucleotides of a circular polyribonucleotide or oligonucleotides may enhance immune evasion. The circular polyribonucleotide or may be synthesized and/or modified by methods well established in the art, such as those described in “Current protocols in nucleic acid chemistry,” Beaucage, S. L. et al. (Eds.), John Wiley & Sons, Inc., New York, NY, USA, which is hereby incorporated herein by reference. Modifications include, for example, end modifications, e.g., 5′ end modifications (phosphorylation (mono-, di- and tri-), conjugation, inverted linkages, etc.), 3′ end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. The modified ribonucleotide bases may also include 5-methylcytidine and pseudouridine. In some embodiments, base modifications may modulate expression, immune response, stability, subcellular localization, to name a few functional effects, of the circular polyribonucleotide. In some embodiments, the modification includes a bi-orthogonal nucleotides, e.g., an unnatural base. See for example, Kimoto et al, Chem Commun (Camb), 2017, 53:12309, DOI: 10.1039/c7cc06661a, which is hereby incorporated by reference.

In some embodiments, sugar modifications (e.g., at the 2′ position or 4′ position) or replacement of the sugar one or more ribonucleotides of the circular polyribonucleotide or oligonucleotide may, as well as backbone modifications, include modification or replacement of the phosphodiester linkages. Specific examples of circular polyribonucleotide include, but are not limited to, circular polyribonucleotide including modified backbones or no natural internucleoside linkages such as internucleoside modifications, including modification or replacement of the phosphodiester linkages. Circular polyribonucleotides having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, the circular polyribonucleotide will include ribonucleotides with a phosphorus atom in its internucleoside backbone.

Modified circular polyribonucleotide or oligonucleotide backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the circular polyribonucleotide may be negatively or positively charged.

The modified nucleotides, which may be incorporated into the circular polyribonucleotide or oligonucleotide, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The a-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked to the circular polyribonucleotide is expected to reduce the innate immune response through weaker binding/activation of cellular innate immune molecules.

In specific embodiments, a modified nucleoside includes an alpha-thio-nucleoside (e.g., 5′-0-(1-thiophosphate)-adenosine, 5′-0-(I-thiophosphate)-cytidine (a-thio-cytidine), 5′-0-(I-thiophosphate)-guanosine, 5′-0-(I-thiophosphate)-uridine, or 5′-0-(1-thiophosphate)-pseudouridine).

Other internucleoside linkages that may be employed according to the present invention, including internucleoside linkages which do not contain a phosphorous atom, are described herein.

In some embodiments, a circular polyribonucleotide or oligonucleotide may include one or more cytotoxic nucleosides. For example, cytotoxic nucleosides may be incorporated into circular polyribonucleotide, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-l-(tetrahydrofuran-2-yl)pyrimidine-2,4(IH,3H)-dione), troxacitabine, tezacitabine, 2′-deoxy-2′-methylidenecytidine (DMDC), and 6-mercaptopurine. Additional examples include fludarabine phosphate, N4-behenoyl-l-beta-D-arabinofuranosylcytosine, N4-octadecyl-1-beta-D-arabinofuranosylcytosine, N4-palmitoyl-l-(2-C-cyano-2-deoxy-beta-D-arabino-pentofuranosyl) cytosine, and P-4055 (cytarabine 5′-elaidic acid ester).

A circular polyribonucleotide or oligonucleotide may or may not be uniformly modified along the entire length of the molecule. For example, one or more or all types of nucleotides (e.g., naturally-occurring nucleotides, purine or pyrimidine, or any one or more or all of A, G, U, C, 1, pU) may or may not be uniformly modified in the circular polyribonucleotide or oligonucleotides, or in a given predetermined sequence region thereof. In some embodiments, the circular polyribonucleotide or oligonucleotide includes a pseudouridine. In some embodiments, the circular polyribonucleotide or oligonucleotide includes an inosine, which may aid in the immune system characterizing the circular polyribonucleotide as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

In some embodiments, all nucleotides in a circular polyribonucleotide or oligonucleotide (or in a given sequence region thereof) are modified. In some embodiments, the modification may include an m6A, which may augment expression; an inosine, which may attenuate an immune response; pseudouridine, which may increase RNA stability, or translational readthrough (stagger element), an m5C, which may increase stability; and a 2,2,7-trimethylguanosine, which aids subcellular translocation (e.g., nuclear localization).

Different sugar modifications, nucleotide modifications, and/or internucleoside linkages (e.g., backbone structures) may exist at various positions in a circular polyribonucleotide or oligonucleotide. One of ordinary skill in the art will appreciate that the nucleotide analogs or other modification(s) may be located at any position(s) of the circular polyribonucleotide or oligonucleotide, such that the function of the circular polyribonucleotide is not substantially decreased. A modification may also be a non-coding region modification. The circular polyribonucleotide or oligonucleotide may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

### Production Methods

In some embodiments, a circular polyribonucleotide includes a deoxyribonucleic acid sequence that is non-naturally occurring and can be produced using recombinant technology (e.g., derived in vitro using a DNA plasmid), chemical synthesis, or a combination thereof.

It is within the scope of the disclosure that a DNA molecule used to produce an RNA circle can include a DNA sequence of a naturally-occurring original nucleic acid sequence, a modified version thereof, or a DNA sequence encoding a synthetic polypeptide not normally found in nature (e.g., chimeric molecules or fusion proteins). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof.

In some embodiments, a linear primary construct or linear mRNA may be cyclized, or concatemerized to create a circular polyribonucleotide described herein. The mechanism of cyclization or concatemerization may occur through methods such as splint ligation methods. The newly formed 5′-/3′-linkage may be an intramolecular linkage or an intermolecular linkage.

Methods of making circular polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

### Circularization

In some embodiments, a linear polyribonucleotide for circularization may be cyclized, or concatemerized. In some embodiments, the linear polyribonucleotide for circularization may be cyclized in vitro prior to formulation and/or delivery. In some embodiments, the circular polyribonucleotide may be in a mixture with linear polyribonucleotides. In some embodiments, the linear polyribonucleotides have the same nucleic acid sequence as the circular polyribonucleotides.

### Extracellular Circularization

In some embodiments, a linear polyribonucleotide for circularization is cyclized, or concatemerized using a chemical method to form a circular polyribonucleotide. In some chemical methods, the 5′-end and the 3′-end of the nucleic acid (e.g., a linear polyribonucleotide for circularization) includes chemically reactive groups that, when close together, may form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond.

In some embodiments, a DNA or RNA ligase is used to enzymatically link a 5′-phosphorylated nucleic acid molecule (e.g., a linear polyribonucleotide for circularization) to the 3′-hydroxyl group of a nucleic acid (e.g., a linear nucleic acid) forming a new phosphorodiester linkage. In an example reaction, a linear polyribonucleotide for circularization is incubated at 37° C. for 1 hour with 1-10 units of T4 RNA ligase (New England Biolabs, Ipswich, MA) according to the manufacturer's protocol. The ligation reaction may occur in the presence of a linear nucleic acid capable of base-pairing with both the 5′- and 3′-region in juxtaposition to assist the enzymatic ligation reaction. In some embodiments, the ligation is splint ligation. For example, a splint ligase, like SplintR® ligase, can be used for splint ligation, RNA ligase II, T4 RNA ligase, or T4 DNA ligase. For splint ligation, a single stranded polynucleotide (splint), like a single stranded RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular polyribonucleotide.

In some embodiments, a DNA or RNA ligase is used in the synthesis of the circular polynucleotides. In some embodiments, either the 5′- or 3′-end of the linear polyribonucleotide for circularization can encode a ligase ribozyme sequence such that during in vitro transcription, the resultant linear polyribonucleotide for circularization includes an active ribozyme sequence capable of ligating the 5′-end of the linear polyribonucleotide for circularization to the 3′-end of the linear polyribonucleotide for circularization. The ligase ribozyme may be derived from the Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In some embodiments, a linear polyribonucleotide for circularization is cyclized or concatermerized by using at least one non-nucleic acid moiety. In one aspect, the at least one non-nucleic acid moiety may react with regions or features near the 5′ terminus and/or near the 3′ terminus of the linear polyribonucleotide for circularization in order to cyclize or concatermerize the linear polyribonucleotide for circularization. In another aspect, the at least one non-nucleic acid moiety may be located in or linked to or near the 5′ terminus and/or the 3′ terminus of the linear polyribonucleotide for circularization. The non-nucleic acid moieties contemplated may be homologous or heterologous. As a non-limiting example, the non-nucleic acid moiety may be a linkage such as a hydrophobic linkage, ionic linkage, a biodegradable linkage, and/or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In some embodiments, the linear polyribonucleotide for circularization is synthesized using IVT and an RNA polymerase, where the nucleotide mixture used for IVT may contain an excess of guanosine monophosphate relative to guanosine triphosphate to preferentially produce RNA with a 5′ monophosphate; the purified IVT product may be circularized using a splint DNA.

In some embodiments, a linear polyribonucleotide for circularization is cyclized or concatermerized due to a non-nucleic acid moiety that causes an attraction between atoms, molecular surfaces at, near or linked to the 5′ and 3′ ends of the linear polyribonucleotide for circularization. As a non-limiting example, one or more linear polyribonucleotides for circularization may be cyclized or concatemerized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In some embodiments, a linear polyribonucleotide for circularization may include a ribozyme RNA sequence near the 5′ terminus and near the 3′ terminus. The ribozyme RNA sequence may covalently link to a peptide when the sequence is exposed to the remainder of the ribozyme. In one aspect, the peptides covalently linked to the ribozyme RNA sequence near the 5′ terminus and the 3′ terminus may associate with each other causing a linear polyribonucleotide for circularization to cyclize or concatemerize. In another aspect, the peptides covalently linked to the ribozyme RNA near the 5′ terminus and the 3′ terminus may cause the linear primary construct or linear mRNA to cyclize or concatemerize after being subjected to ligated using various methods known in the art such as, but not limited to, protein ligation. Non-limiting examples of ribozymes for use in the linear primary constructs or linear RNA of the present invention or a non-exhaustive listing of methods to incorporate and/or covalently link peptides are described in US patent application No. US20030082768, the contents of which is here in incorporated by reference in its entirety.

In some embodiments, a linear polyribonucleotide for circularization may include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). In some embodiments, the 5′ end of at least a portion of the linear polyribonucleotides comprises a monophosphate moiety. In some embodiments, the population of polyribonucleotides including circular and linear polyribonucleotides is contacted with RppH prior to digesting at least a portion of the linear polyribonucleotides with a 5′ exonuclease and/or a 3′ exonuclease. Alternately, converting the 5′ triphosphate of the linear polyribonucleotide for circularization into a 5′ monophosphate may occur by a two-step reaction including: (a) contacting the 5′ nucleotide of the linear polyribonucleotide for circularization with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization efficiency of the circularization methods provided herein is at least about 40%. In some embodiments, the circularization method provided has a circularization efficiency of between about 10% and about 100%; for example, the circularization efficiency may be about 15%, about 20%, about 25%, about 30%, about 35%, about 40%, about 45%, about 50%, about 55%, about 60%, about 65%, about 70%, about 75%, about 80%, about 85%, about 90%, about 95%, and about 99%. In some embodiments, the circularization efficiency is between about 20% and about 80%. In some embodiments, the circularization efficiency is between about 30% and about 60%. In some embodiments the circularization efficiency is about 40%.

In some embodiments, the circular polyribonucleotide includes an internal splicing element that when replicated the spliced ends are joined together. Some examples may include miniature introns (<100 nt) with splice site sequences and short inverted repeats (30-40 nt) such as AluSq2, AluJr, and AluSz, inverted sequences in flanking introns, A/u elements in flanking introns, and motifs found in (suptable4 enriched motifs) cis-sequence elements proximal to backsplice events such as sequences in the 200 bp preceding (upstream of) or following (downstream from) a backsplice site with flanking exons. In some embodiments, the circular polyribonucleotide includes at least one repetitive nucleotide sequence described elsewhere herein as an internal splicing element. In such embodiments, the repetitive nucleotide sequence may include repeated sequences from the Alu family of introns. In some embodiments, a splicing-related ribosome binding protein can regulate circular polyribonucleotide biogenesis (e.g. the Muscleblind and Quaking (QKI) splicing factors).

In some embodiments, the circular polyribonucleotide may include canonical splice sites that flank head-to-tail junctions of the circular polyribonucleotide.

In some embodiments, the circular polyribonucleotide may include a bulge-helix-bulge motif, comprising a 4-base pair stem flanked by two 3-nucleotide bulges. Cleavage occurs at a site in the bulge region, generating characteristic fragments with terminal 5′-hydroxyl group and 2′, 3′-cyclic phosphate. Circularization proceeds by nucleophilic attack of the 5′—OH group onto the 2′, 3′-cyclic phosphate of the same molecule forming a 3′, 5′-phosphodiester bridge.

In some embodiments, the circular polyribonucleotide may include a multimeric repeating RNA sequence that harbors a HPR element. The HPR comprises a 2′,3′-cyclic phosphate and a 5′-OH termini. The HPR element self-processes the 5′- and 3′-ends of the linear circular polyribonucleotide, thereby ligating the ends together.

In some embodiments, the circular polyribonucleotide may include a sequence that mediates self-ligation. In one embodiment, the circular polyribonucleotide may include a HDV sequence (e.g., HDV replication domain conserved sequence, GGCUCAUCUCGACAAGAGGCGGCAGUCCUCAGUACUCUUACUCUUUUCUGUAAAGAGGAGACUG CUGGACUCGCCGCCCAAGUUCGAGCAUGAGCC (Beeharry et al 2004) (SEQ ID NO: 24) or GGCUAGAGGCGGCAGUCCUCAGUACUCUUACUCUUUUCUGUAAAGAGGAGACUGCUGGACUCGC CGCCCGAGCC (SEQ ID NO: 25))) to self-ligate. In one embodiment, the circular polyribonucleotide may include loop E sequence (e.g. in PSTVd) to self-ligate. In another embodiment, the circular polyribonucleotide may include a self-circularizing intron, e.g., a 5′ and 3′ slice junction, or a self-circularizing catalytic intron such as a Group I, Group II or Group III Introns. Nonlimiting examples of group I intron self-splicing sequences may include self-splicing permuted intron-exon sequences derived from T4 bacteriophage gene td, and the intervening sequence (IVS) rRNA of Tetrahymena.

### Other Circularization Methods

In some embodiments, linear polyribonucleotides for circularization may include complementary sequences, including either repetitive or nonrepetitive nucleic acid sequences within individual introns or across flanking introns. Repetitive nucleic acid sequence are sequences that occur within a segment of the circular polyribonucleotide. In some embodiments, the circular polyribonucleotide includes a repetitive nucleic acid sequence. In some embodiments, the repetitive nucleotide sequence includes poly CA or poly UG sequences. In some embodiments, the circular polyribonucleotide includes at least one repetitive nucleic acid sequence that hybridizes to a complementary repetitive nucleic acid sequence in another segment of the circular polyribonucleotide, with the hybridized segment forming an internal double strand. In some embodiments, the circular polyribonucleotide includes between 1 and 10 (e.g., 2, 3, 4, 5, 6, 7, 8, 9, and 10) repetitive nucleic acid sequences that hybridize to a complementary repetitive nucleic acid sequence in another segment of the circular polyribonucleotide, with the hybridized segment forming an internal double strand. In some embodiments, the circular polyribonucleotide includes 2 repetitive nucleic acid sequences that hybridize to a complementary repetitive nucleic acid sequence in another segment of the circular polyribonucleotide, with the hybridized segment forming an internal double strand. In some embodiments, repetitive nucleic acid sequences and complementary repetitive nucleic acid sequences from two separate circular polyribonucleotides hybridize to generate a single circularized polyribonucleotide, with the hybridized segments forming internal double strands. In some embodiments, the complementary sequences are found at the 5′ and 3′ ends of the linear polyribonucleotides for circularization. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the circular polyribonucleotide. Such methods may include, but are not limited to click chemistry (e.g., alkyne and azide based methods, or clickable bases), olefin metathesis, phosphoramidate ligation, hemiaminal-imine crosslinking, base modification, and any combination thereof.

In some embodiments, enzymatic methods of circularization may be used to generate the circular polyribonucleotide. In some embodiments, a ligation enzyme, e.g., DNA or RNA ligase, may be used to generate a template of the circular polyribonuclease or complement, a complementary strand of the circular polyribonuclease, or the circular polyribonuclease.

Circularization of the circular polyribonucleotide may be accomplished by methods known in the art, for example, those described in “RNA circularization strategies in vivo and in vitro” by Petkovic and Muller from Nucleic Acids Res, 2015, 43(4): 2454-2465, and “In vitro circularization of RNA” by Muller and Appel, from RNA Biol, 2017, 14(8):1018-1027.

The circular polyribonucleotide may encode a sequence and/or motif useful for replication. Exemplary replication elements are described in paragraphs [0280]-[0286] of International Patent Publication No. WO2019/118919, which is hereby incorporated by reference in its entirety.

In some embodiments, linear circular polyribonucleotides may include complementary sequences, including either repetitive or nonrepetitive nucleic acid sequences within individual introns or across flanking introns. Repetitive nucleic acid sequence are sequences that occur within a segment of the circular polyribonucleotide. In some embodiments, the circular polyribonucleotide includes a repetitive nucleic acid sequence. In some embodiments, the repetitive nucleotide sequence includes poly CA or poly UG sequences. In some embodiments, the circular polyribonucleotide includes at least one repetitive nucleic acid sequence that hybridizes to a complementary repetitive nucleic acid sequence in another segment of the circular polyribonucleotide, with the hybridized segment forming an internal double strand. In some embodiments, repetitive nucleic acid sequences and complementary repetitive nucleic acid sequences from two separate circular polyribonucleotides hybridize to generate a single circularized polyribonucleotide, with the hybridized segments forming internal double strands. In some embodiments, the complementary sequences are found at the 5′ and 3′ ends of the linear circular polyribonucleotides. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the circular polyribonucleotide. Such methods may include, but are not limited to click chemistry (e.g., alkyne and azide based methods, or clickable bases), olefin metathesis, phosphoramidate ligation, hemiaminal-imine crosslinking, base modification, and any combination thereof.

### Replication Elements

The circular polyribonucleotide may encode a sequence and/or motifs useful for replication. Replication of a circular polyribonucleotide may occur by generating a complement circular polyribonucleotide. In some embodiments, the circular polyribonucleotide includes a motif to initiate transcription, where transcription is driven by either endogenous cellular machinery (DNA-dependent RNA polymerase) or an RNA-depended RNA polymerase encoded by the circular polyribonucleotide. The product of rolling-circle transcriptional event may be cut by a ribozyme to generate either complementary or propagated circular polyribonucleotide at unit length. The ribozymes may be encoded by the circular polyribonucleotide, its complement, or by an RNA sequence in trans. In some embodiments, the encoded ribozymes may include a sequence or motif that regulates (inhibits or promotes) activity of the ribozyme to control circular RNA propagation. In some embodiments, unit-length sequences may be ligated into a circular form by a cellular RNA ligase. In some embodiments, the circular polyribonucleotide includes a replication element that aids in self amplification. Examples of such replication elements include, but are not limited to, HDV replication domains described elsewhere herein, RNA promotor of Potato Spindle Tuber Viroid (see for example Kolonko 2005 Virology), and replication competent circular RNA sense and/or antisense ribozymes such as antigenomic 5′-CGGGUCGGCAUGGCAUCUCCACCUCCUCGCGGUCCGACCUGGGCAUCCGAAGGAGGACGCACG UCCACUCGGAUGGCUAAGGGAGAGCCA-3′ (SEQ ID NO: 26) or genomic 5′-UGGCCGGCAUGGUCCCAGCCUCCUCGCUGGCGCCGGCUGGGCAACAUUCCGAGGGGACCGUCC CCUCGGUAAUGGCGAAUGGGACCCA-3′ (SEQ ID NO: 27).

In some embodiments, the circular polyribonucleotide includes at least one stagger element as described herein to aid in replication. A stagger element within the circular polyribonucleotide can cleave long transcripts replicated from the circular polyribonucleotide to a specific length that could subsequently circularize to form a complement to the circular polyribonucleotide.

In another embodiment, the circular polyribonucleotide includes at least one ribozyme sequence to cleave long transcripts replicated from the circular polyribonucleotide to a specific length, where another encoded ribozyme cuts the transcripts at the ribozyme sequence. Circularization forms a complement to the circular polyribonucleotide.

In some embodiments, the circular polyribonucleotide is substantially resistant to degradation, e.g., by exonucleases.

In some embodiments, the circular polyribonucleotide replicates within a cell. In some embodiments, the circular polyribonucleotide replicates within in a cell at a rate of between about 10%-20%, 20%-30%, 30%-40%, 40%-50%, 50%-60%, 60%-70%, 70%-75%, 75%-80%, 80%-85%, 85%-90%, 90%-95%, 95%-99%, or any percentage therebetween. In some embodiments, the circular polyribonucleotide is replicated within a cell and is passed to daughter cells. In some embodiments, a cell passes at least one circular polyribonucleotide to daughter cells with an efficiency of at least 25%, 50%, 60%, 70%, 80%, 85%, 90%, 95%, or 99%. In some embodiments, cell undergoing meiosis passes the circular polyribonucleotide to daughter cells with an efficiency of at least 25%, 50%, 60%, 70%, 80%, 85%, 90%, 95%, or 99%. In some embodiments, a cell undergoing mitosis passes the circular polyribonucleotide to daughter cells with an efficiency of at least 25%, 50%, 60%, 70%, 80%, 85%, 90%, 95%, or 99%.

In some embodiments, the circular polyribonucleotide replicates within the host cell. In one embodiment, the circular polyribonucleotide is capable of replicating in a mammalian cell, e.g., human cell.

While in some embodiments the circular polyribonucleotide replicates in the host cell, the circular polyribonucleotide does not integrate into the genome of the host, e.g., with the host's chromosomes. In some embodiments, the circular polyribonucleotide has a negligible recombination frequency, e.g., with the host's chromosomes. In some embodiments, the circular polyribonucleotide has a recombination frequency, e.g., less than about 1.0 cM/Mb, 0.9 cM/Mb, 0.8 cM/Mb, 0.7 cM/Mb, 0.6 cM/Mb, 0.5 cM/Mb, 0.4 cM/Mb, 0.3 cM/Mb, 0.2 cM/Mb, 0.1 cM/Mb, or less, e.g., with the host's chromosomes.

### Detection of Linear and Circular RNA

The presence of linear RNA in pharmaceutical circular RNA preparations can have unexpected and sometimes undesirable effects. Thus, circular RNAs may be enriched, separated, and/or purified relative to linear RNA; methods (e.g., methods of manufacturing circular RNA preparations) whereby linear RNAs can be monitored, evaluated and/or controlled; and methods of using such pharmaceutical compositions and preparations. In some embodiments, a circular RNA preparation has no more than a threshold level of linear RNA, e.g., a circular RNA preparation is enriched over linear RNA or purified to reduce linear RNA. In some embodiments, the circular polyribonucleotide population is enriched such that a mixture of circular polyribonucleotides and linear polyribonucleotides includes a threshold level of circular polyribonucleotides.

Generally, detection and quantitation of an element in a pharmaceutical preparation includes the use of a reference standard that is either the component of interest (e.g., circular RNA, linear RNA, fragment, impurity, etc.) or is a similar material (e.g., using a linear RNA structure of the same sequence as a circular RNA structure as a standard for circular RNA), or includes the use of an internal standard or signal from a test sample. In some embodiments, the standard is used to establish the response from a detector for a known or relative amount of material (response factor). In some embodiments, the response factor is determined from a standard at one or multiple concentrations (e.g., using linear regression analysis). In some embodiments, the response factor is then used to determine the amount of the material of interest from the signal due to that component. In some embodiments, the response factor is a value of one or is assumed to have a value of one.

In some embodiments, detection, and quantification of linear versus circular RNA in the pharmaceutical composition is determined using a comparison to a linear version of the circular polyribonucleotides. In some embodiments, the mass of total ribonucleotide in the pharmaceutical composition is determined using a standard curve generated using a linear version of the circular polyribonucleotide and assuming a response factor of one. In some embodiments, a w/w percentage of circular polyribonucleotide in the pharmaceutical preparation is determined by a comparison of a standard curve generated by band intensities of multiple known amounts of a linear version of the circular polyribonucleotide to a band intensity of the circular polyribonucleotide in the pharmaceutical preparation. In some embodiments, the bands are produced during gel-base electrophoresis, and the band intensities are measured by a gel imager (e.g., an E-gel Imager). In some embodiments, a circular polyribonucleotide preparation includes less than a threshold amount (e.g., where the threshold amount is a reference criterion, e.g., a pharmaceutical release specification for the circular polyribonucleotide preparation) of linear polyribonucleotide molecules when evaluated as described herein.

In some embodiments, detection, and quantification of nicked versus total RNA in the pharmaceutical composition is determined by sequencing after gel extraction of the preparation including the circular RNA. In some embodiments, detection, and quantification of nicked versus linear RNA in the pharmaceutical composition is determined by sequencing after gel extraction of the preparation including the circular RNA. In some embodiments, a circular polyribonucleotide preparation includes less than a threshold amount (e.g., where the threshold amount is a reference criterion, e.g., a pharmaceutical release specification for the circular polyribonucleotide preparation) of nicked RNA, linear RNA, or combined linear and nicked RNA when evaluated as described herein. For example, the reference criterion for the amount of linear polyribonucleotide molecules present in the preparation is no more than 30%, 20%, 15%, 10%, 1%, 0.5%, or 0.1% linear polyribonucleotide molecules, or any percentage therebetween, relative to total ribonucleotide molecules in the preparation. In some embodiments, the reference criterion for the amount of nicked polyribonucleotide molecules present in the preparation is no more than 30%, 20%, 15%, 10%, 1%, 0.5%, or 0.1%, or any percentage therebetween, nicked polyribonucleotide molecules relative to total ribonucleotide molecules in the preparation. In some embodiments, the reference criterion for the amount of linear and nicked polyribonucleotide molecules present in the preparation is no more than 40%, 30%, 20%, 15%, 10%, 1%, 0.5%, or 0.1%, or any percentage therebetween, combined linear polyribonucleotide and nicked polyribonucleotide molecules relative to total ribonucleotide molecules in the preparation.

In some embodiments, the standard is run under the same conditions as the sample. For example, the standard is run with the same type of gel, same buffer, and same exposure as the sample. In further embodiments, the standard is run in parallel with the sample. In some embodiments, a quantification of an element is repeated (e.g., twice or in triplicate) in a plurality of samples from the subject preparation to obtain a mean result. In some embodiments, quantitation of a linear RNA is measured using parallel capillary electrophoresis (e.g., using a Fragment Analyzer or analytical HPLC with UV detection).

Alternatively, to measure the amount of linear and circular RNA present, qPCR reverse transcription (RT-qPCR) using two sets of primers: one across the ligation site and one specific to the ORF. The primers across the ligation site report on the levels of circular RNA whereas the primers specific to the ORF report on both circular and linear RNA. In some embodiments, the reverse transcription reactions are performed with a reverse transcriptase (e.g., Super-Script II: RNase H, Invitrogen) and random hexamers in accordance with the manufacturer's instruction. In some embodiments, the amplified PCR products are analyzed using polyacrylamide gel electrophoresis and visualized by ethidium bromide staining. In some embodiments, to estimate the enrichment factor of the circular polyribonucleotides, the PCR products may quantified using densitometry and the concentrations of total RNA samples may be measured by UV absorbance.

### OTHER EMBODIMENTS

Various modifications and variations of the described compositions, methods, and uses of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

All publications, patents, and patent applications are herein incorporated by reference in their entirety to the same extent as if each individual publication, patent, or patent application was specifically and individually indicated to be incorporated by reference in its entirety.

### Examples

The following examples, which are intended to illustrate, rather than limit, the disclosure, are put forth to provide those of ordinary skill in the art with a description of how the compositions and methods described herein may be used, made, and evaluated. The examples are intended to be purely exemplary of the disclosure and are not intended to limit the scope of what the inventors regard as their invention.

### Example 1: Optimization of Exonuclease Concentrations for Degradation of Linear Polyribonucleotides

This example demonstrates that 5′ and 3′ exonucleases can be used to degrade linear polyribonucleotides.

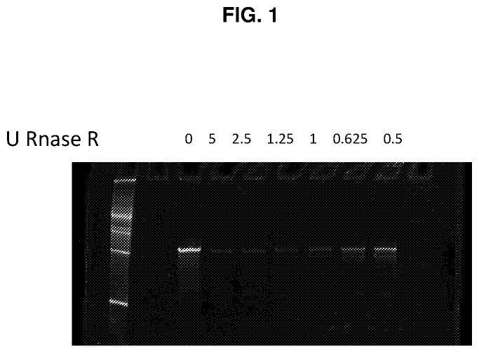

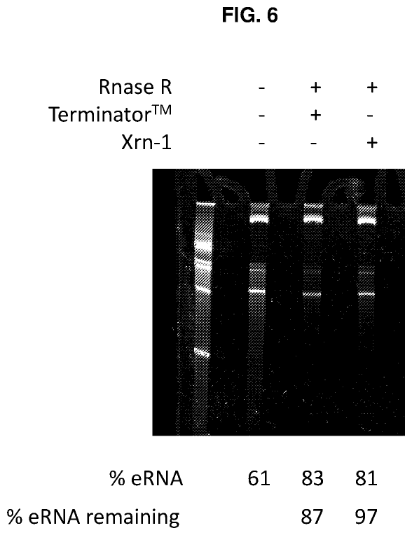

In this experiment, 2.5 μg of linear polyribonucleotide encoding Gaussia Luciferase (gLuc) was digested with 0 U, 0.5 U, 0.625 U, 1 U, 1.25 U, 2.5 U or 5 U of RNase R, a 3′ exonuclease, at 37° C. for 90 minutes. Each digestion was performed in Terminator™ A buffer (Lucigen), quenched with EDTA and analyzed by gel electrophoresis. The TBE-Urea gel was stained with SYBR Safe and imaged on an Bright imager (Invitrogen). The results from this experiment show that the linear polyribonucleotide was degraded with the 3′ exonuclease RNase R (FIG. 1). 0.5 U of RNase R per 1 μg of RNA was selected and used in Examples 5 and 7 (FIGS. 6, 8-10).

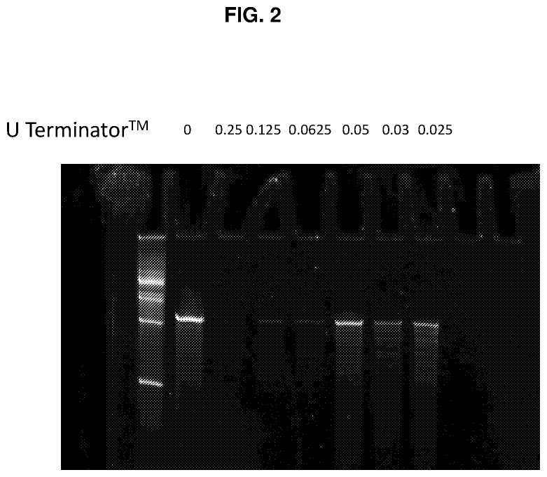

In this experiment, 2.5 μg of linear polyribonucleotide encoding gLuc was digested with 0 U, 0.025 U, 0.03 U, 0.05 U, 0.0625 U, 0.125 U or 0.25 U of Terminator™ exonuclease, a 5′ exonuclease, at 37° C. for 60 minutes. Each digestion was performed in Terminator™ A buffer (Lucigen), quenched with EDTA and analyzed by gel electrophoresis. The TBE-Urea gel was stained with SYBR Safe and imaged on an iBright imager (Invitrogen). The results from this experiment show that the linear polyribonucleotide was degraded with the Terminator™ 5′ exonuclease (FIG. 2). 0.025 U of Terminator™ exonuclease per 1 μg of RNA was selected and used in Examples 5 and 7 (FIGS. 6, 8-10).

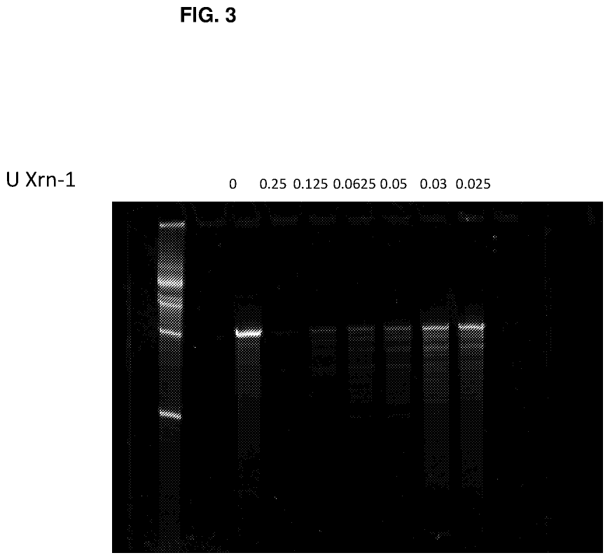

In this experiment, 2.5 μg of linear polyribonucleotide encoding gLuc was digested with 0 U, 0.025 U, 0.03 U, 0.05 U, 0.0625 U, 0.125 U or 0.25 U of Xrn-1, a 5′ exonuclease, at 37° C. for 60 minutes. Each digestion was performed in Terminator™ A buffer (Lucigen), quenched with EDTA and analyzed by gel electrophoresis. The TBE-Urea gel was stained with SYBR Safe and imaged on an iBright imager (Invitrogen). The results from this experiment show that the linear polyribonucleotide was degraded with the 5′ exonuclease Xrn-1 (FIG. 3). 0.05 U of Xrn-1 per 1 μg of RNA was selected and used in Examples 5 and 7 (FIGS. 6, 8-10)

This Example 1 demonstrated that 5′ and 3′ exonucleases degrade linear polyribonucleotides.

### Example 2: In Vitro Production of Circular Polyribonucleotide Encoding Gaussia Luciferase

This example describes in vitro production of the circular polyribonucleotide.

The circular polyribonucleotide was designed with an IRES and an ORF encoding Gaussia Luciferase (GLuc), and two spacer elements flanking the IRES-ORF. The circular polyribonucleotide was generated using in vitro synthesis (IVT) using one of the two following methods.

In the first method, unmodified linear polyribonucleotide was synthesized by IVT using T7 RNA polymerase from a DNA segment with the above elements. Transcribed polyribonucleotide was purified with an RNA purification system (New England Biolabs, Inc.), treated with RNA 5′ phosphohydrolase (RppH) (New England Biolabs, M0356) following the manufacturer's instructions, and purified again with the RNA purification system. RppH-treated linear polyribonucleotide was circularized using a splint DNA.

In the second method, unmodified linear polynucleotide was synthesized by IVT using T7 RNA polymerase from a DNA segment with the above elements. The nucleotide mixture used for IVT contained an excess of guanosine monophosphate relative to guanosine triphosphate to preferentially produce RNA with a 5′ monophosphate. Transcribed polyribonucleotide was purified with an RNA purification system (New England Biolabs, Inc.) following the manufacturer's instruction. The purified IVT product was circularized using a splint DNA.

Circular polyribonucleotide was generated by splint-ligation as follows: Transcribed linear polyribonucleotide and a DNA splint (5′-GTTTTTCGGCTATTCCCAATAGCCGTTTTG-3′; SEQ ID NO: 1) were mixed and annealed and treated with an RNA ligase to produce a post-ligation polyribonucleotide mixture. A by-product of the circularization reaction is unreacted linear polyribonucleotides.

### Example 3: Selective Degradation of Linear Polyribonucleotides Using a Combination of 5′ and 3′ Exonucleases

This example demonstrates that a combination of 5′ and 3′ exonucleases can be used to selectively degrade linear polyribonucleotides.

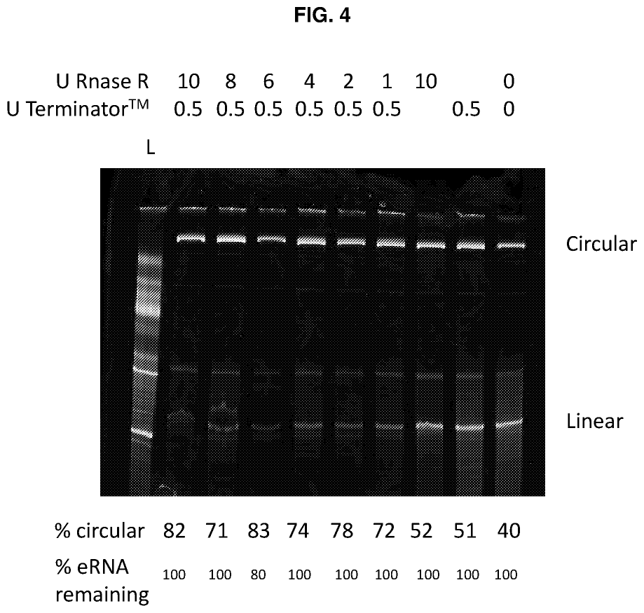

To degrade the unreacted linear polyribonucleotides after the circularization reaction, a combination of a 5′ exonuclease and a 3′ exonuclease was used. In this Example, 2.5 μg of the post-ligation polyribonucleotide mixture including both circular and linear polyribonucleotides produced as described in Example 2, was digested concomitantly with 0.5 U of Terminator™ 5′ exonuclease and 1 U, 2 U, 4 U, 6 U, 8 U or 10 U of the 3′ exonuclease RNase R at 37° C. for 90 minutes. Each digestion was performed in Terminator™ A buffer (Lucigen), quenched with EDTA and analyzed by gel electrophoresis. The TBE-Urea gel was stained with SYBR Safe and imaged on an iBright imager (Invitrogen). The percentage of the total polyribonucleotides after digestion with the exonuclease that are circular polyribonucleotides (% circular), and the percentage of the circular polyribonucleotides that are remaining after digesting with the exonuclease compared the amount of circular polyribonucleotides that were present post-ligation (% eRNA remaining) were analyzed by Image Lab. The results of this Example show that a combination of 5′ and 3′ exonucleases selectively degraded linear polyribonucleotides in the post-ligation polyribonucleotide mixture (FIG. 4).

### Example 4: Enrichment of Circular Polyribonucleotide Using a Combination of 5′ and 3′ Exonucleases

This example demonstrates that circular polyribonucleotide can be enriched using a combination of 5′ and 3′ exonucleases in a scaled up reaction.

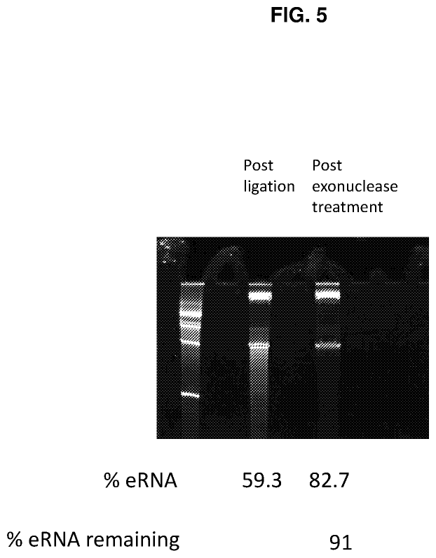

In this Example, 1 mg of the post-ligation polyribonucleotide mixture produced as described in Example 2, was digested concomitantly with 4000 U of RNase R and 200 U of Terminator™ exonuclease at 37° C. for 60 minutes. The digestion was performed in Terminator™ A buffer, quenched with EDTA and analyzed by gel electrophoresis. The TBE-Urea gel was stained with SYBR Safe and imaged on an iBright imager (Invitrogen). The percentage of the total polyribonucleotides after digestion with the exonuclease that are circular polyribonucleotides (% circular), and the percentage of the circular polyribonucleotides that are remaining after digesting with the exonuclease compared the amount of circular polyribonucleotides that were present post-ligation (% eRNA remaining) were analyzed by Image Lab. The results of this Example show that circular polyribonucleotide in the post-ligation polyribonucleotide mixture was enriched using a combination 5′ and 3′ exonucleases (FIG. 5).

### Example 5: Enrichment of Circular Polyribonucleotide Using Decreased Concentrations of 5′ and 3′ Exonucleases

This example demonstrates that circular polyribonucleotide can be enriched using decreased concentrations of a combination of 5′ and 3′ exonucleases.

In this Example, 2.5 μg of the post-ligation polyribonucleotide mixture produced as described in Example 2, was digested concomitantly with either (i) 1.25 U of RNase R and 0.0625 U of Terminator™ exonuclease; or (ii) 1.25 U RNase R and 0.0625 U of Xrn-1 at 37° C. for 60 minutes in Terminator™ A buffer. Each digestion was quenched with EDTA and analyzed by gel electrophoresis. The TBE-Urea gel was stained with SYBR Safe and imaged on an Bright imager (Invitrogen The percentage of the total polyribonucleotides after digestion with the exonuclease that are circular polyribonucleotides (% circular), and the percentage of the circular polyribonucleotides that are remaining after digesting with the exonuclease compared the amount of circular polyribonucleotides that were present post-ligation (% eRNA remaining) were analyzed by Image Lab. The results of this Example show that digestion concomitantly with 5′ and 3′ exonucleases in optimized concentrations selectively degraded linear polyribonucleotides in the post-ligation polyribonucleotide mixture, producing an enriched circular polyribonucleotide preparation (FIG. 6).

### Example 6: Enrichment of Circular Polyribonucleotide Using a Combination of 5′ and 3′ Exonucleases

This example compares two different combinations of 5′ and 3′ exonucleases used to enrich for circular polyribonucleotide.

In this Example, 2.5 μg of the post-ligation polyribonucleotide mixture produced as described in Example 2, was digested concomitantly with 0.5 U of RNase R (Lucigen or MC Labs) and 0.025 U of Terminator™ (Lucigen) exonuclease per 1 μg of RNA; or (ii) 0.5 U of RNase R (Lucigen or MC Labs) and 0.2 U of Xrn-1 (New England Biolabs) per 1 μg of RNA, at 37° C. for 60 minutes in Terminator™ A buffer. Each digestion was quenched with EDTA and analyzed by gel electrophoresis. The TBE-Urea gel was stained with SYBR Safe and imaged on an iBright imager (Invitrogen). The percentage of the total polyribonucleotides after digestion with the exonuclease that are circular polyribonucleotides (% circular), and the percentage of the circular polyribonucleotides that are remaining after digesting with the exonuclease compared the amount of circular polyribonucleotides that were present post-ligation (% eRNA remaining) were analyzed by Image Lab.

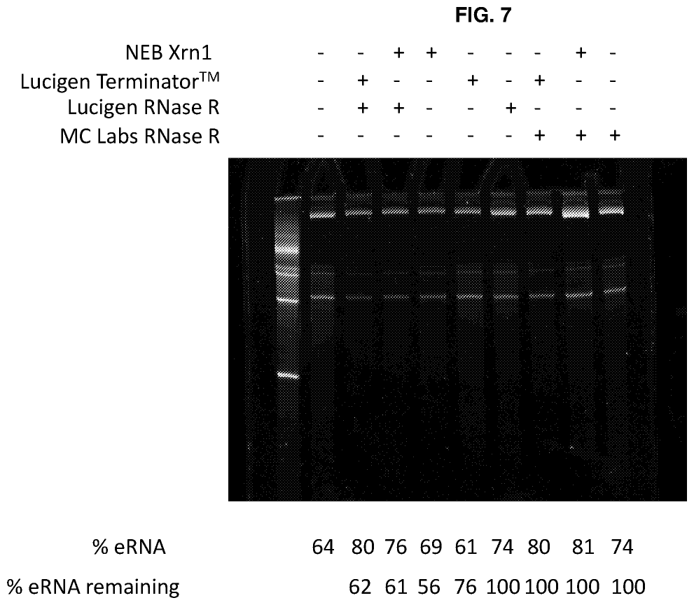

FIG. 7 shows the results of this Example (lane 1: NEB ssRNA ladder; lane 2: 2.5 μg untreated post-ligation mixture; lane 3: 2.5 μg of post-ligation mixture treated with RNase R+Terminator™ exonuclease; lane 4: 2.5 μg of post-ligation mixture treated with RNase R (Lucigen)+Xrn-1 exonuclease; lane 5: 2.5 μg of post-ligation mixture treated with Xrn-1; lane 6: 2.5 μg of post-ligation mixture treated with Terminator™ alone; lane7: 2.5 μg post-ligation mixture treated with RNase R (Lucigen) alone; lane 8: 2.5 μg post-ligation mixture treated with Terminator™ exonuclease and RNase R (MC Labs); lane 9: 2.5 μg post ligation mixture mixture treated with Xrn-1 and RNase R (MC Labs); lane: 10: 2.5 μg post ligation mixture treated with RNase R (MC Labs)). The post-ligation polyribonucleotide mixture consisted of 64% circular polyribonucleotide (lane 2). After digestion with RNase R and Terminator™ exonucleases, the circular polyribonucleotide in the post-ligation polyribonucleotide mixture was enriched to 80% (lane 3 & lane 8) circular polyribonucleotide. After digestion with RNase R+Xrn-1, the circular polyribonucleotide in the post-ligation polyribonucleotide mixture was enriched to 76% (lane 4) with Lucigen RNase R and 81% (lane 9) with MC Labs RNase R. When only Xrn-1 or Terminator™ exonuclease was used alone, the post-ligation mixture of circular polyribonucleotides was enriched to 69% (lane 5) or 74% (lane 7) respectively. When only Lucigen RNase R or the MC Labs RNase R was used alone, the post-ligation mixture of circular polyribonucleotides was enriched to 61% (lane 6) or 74% (lane 10) respectively.

### Example 7. Identification of Exonuclease Reaction Time for the Enrichment of Circular Polyribonucleotides

This example compares reaction times of the digestion of linear polyribonucleotides with a 5′ and 3′ exonuclease used to enrich for circular polyribonucleotides.

In this experiment, 3.75 μg of post-ligation polyribonucleotide mixture produced as described in Example 2, was digested concomitantly with either (i) 15 U of RNase R and 1.5 U of Terminator™ exonuclease in Terminator™ A buffer: The reaction was performed at 37° C. for 15, 30, 45, 60, 75, 90, 105, or 120 minutes. Each digestion was quenched with EDTA and analyzed by gel electrophoresis. The TBE-Urea gel was stained with SYBR Safe and imaged on an Bright imager (Invitrogen). The percentage of circular polyribonucleotide was analyzed by Image Lab. The results of this Example show that reaction times ≤90 and minutes resulted in at least 50% of the circular polyribonucleotides remaining post-ligation and before digestion with an exonuclease to be still remaining after digestion with both the 5′ and 3′ exonucleases. The results of this Example also show that a reaction time ≥15 minutes results in the total resulting poly ribonucleoties to include at least 80% circular polyribonucleotides. The results of each reaction time tested are summarized in Table 2.

TABLE 2
Enrichment of circular polyribonucleotide after varying reaction
time of digest with 5′ and 3′ exonucleases

| Reaction | % (w/w) of total | % circular RNA remaining after |
|---|---|---|
| time | polyribonucleotides | digestion with exonuclease |
| (minutes) | that are circular | compared to before |
| 0 | 68 | — |
| 15 | 82 | 86 |
| 30 | 88 | 72 |
| 45 | 87 | 76 |
| 60 | 87 | 57 |
| 75 | 89 | 53 |
| 90 | 91 | 50 |
| 105 | 93 | 47 |
| 120 | 92 | 44 |

### Example 8: Identification of Buffer Conditions for Enrichment of Circular Polyribonucleotide

This example describes the identification of buffer conditions for enrichment of circular polyribonucleotide using combinations of 5′ and 3′ exonucleases.

### Experiment 1

In this experiment, 2.5 μg of post-ligation polyribonucleotide mixture produced as described in Example 2, was digested concomitantly with either (i) 1.25 U of RNase R and 0.0625 U of Terminator™ exonuclease; or (ii) 1.25 U of RNase R and 0.0625 U of Xrn-1, at 37° C. for 60 minutes in one of the following buffers:
Buffer T: Terminator™ A buffer (Lucigen; proprietary)
Buffer 1: 100 mM NaCl, 50 mM Tris-HCl (pH 8)
Buffer 2: 100 mM NaCl, 50 mM Tris-HCl (pH 8), 0.5 mM MgCl₂
Buffer 3: 100 mM NaCl, 50 mM Tris-HCl (pH 8), 1 mM DTT
Buffer 4: 100 mM NaCl, 50 mM Tris-HCl (pH 8), 0.5 mM MgCl₂, 1 mM DTT

As controls, 2.5 μg of post-ligation polyribonucleotide mixture produced as described in Example 2, was also mixed in Buffer 1, Buffer 2, Buffer 3 or Buffer 4, without exonuclease digestion (buffer controls). Each digestion was quenched with EDTA and analyzed by gel electrophoresis. The TBE-Urea gel was stained with SYBR Safe and imaged on an iBright imager (Invitrogen). The percentage of the total polyribonucleotides after digestion with the exonuclease that are circular polyribonucleotides (% circular), and the percentage of the circular polyribonucleotides that are remaining after digesting with the exonuclease compared the amount of circular polyribonucleotides that were present post-ligattion (% eRNA remaining) were analyzed by Image Lab.

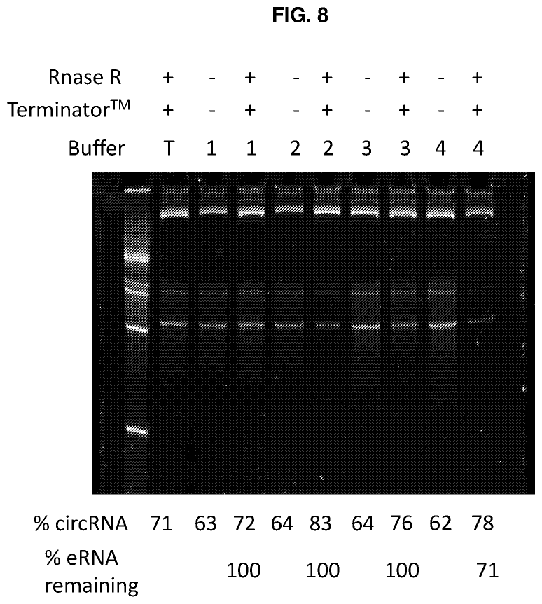

FIG. 8 shows the results of the digestions with the combination of Terminator™ exonuclease and RNase R in various buffers and corresponding buffer controls (lane 1: NEB ssRNA ladder; lane 2: Terminator™ A buffer (Terminator™+RNase R); lane 3: Buffer 1 control (no exonuclease digestions); lane 4: Buffer 1 (Terminator™+RNase R); lane 5: Buffer 2 control (no exonuclease digestions); lane 6: Buffer 2 (Terminator™+RNase R); lane 7: Buffer 3 control (no exonuclease digestions); lane 8: Buffer 3 (Terminator™+RNase R); lane 9: Buffer 4 control (no exonuclease digestions); lane 10: Buffer 4 (Terminator™+RNase R)). The circular polyribonucleotide in the post-ligation polyribonucleotide mixture was enriched to 83% after digestion with the combination of Terminator™ exonuclease and RNase R in Buffer 2 (lane 6).

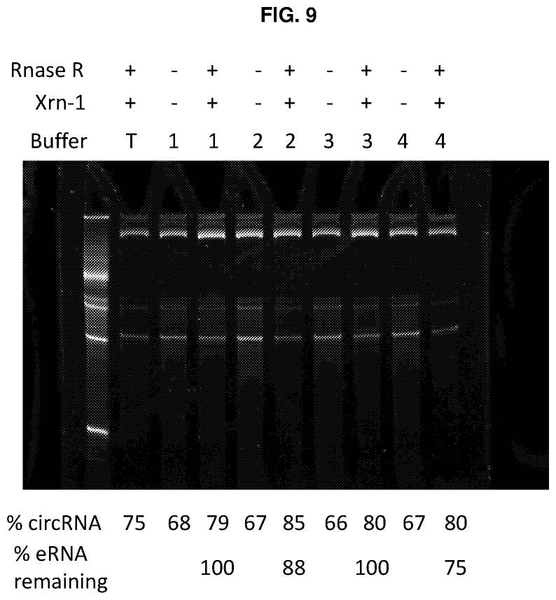

FIG. 9 shows the results of the digestions with the combination of Xrn-1 and RNase R in various buffers and corresponding buffer controls (lane 1: NEB ssRNA ladder; lane 2: Terminator™ A buffer (Xrn-1+RNase R); lane 3: Buffer 1 control (no exonuclease digestions); lane 4: Buffer 1 (Xrn-1+RNase R); lane 5: Buffer 2 control (no exonuclease digestions); lane 6: Buffer 2 (Xrn-1+RNase R); lane 7: Buffer 3 control (no exonuclease digestions); lane 8: Buffer 3 (Xrn-1+RNase R); lane 9: Buffer 4 control (no exonuclease digestions); lane 10: Buffer 4 (Xrn-1+RNase R)). These results show that the circular polyribonucleotide in the post-ligation polyribonucleotide mixture was enriched to 85% after digestion with the combination of Xrn-1 and RNase R in Buffer 2 (lane 6).

### Experiment 2

In this experiment, 2.5 μg of post-ligation polyribonucleotide mixture prepared as described in Example 2, was digested concomitantly 0.0625 U of Xrn-1 and 1.25 U RNase R at 37° C. for 60 minutes in one of the following buffers:
Terminator™ A buffer (Lucigen; proprietary)
Xrn-1 buffer (100 mM NaCl, 50 mM Tris-HCl, 10 mM MgCl₂, 1 mM DTT; pH 7.9)
RNase R buffer (Lucigen) (100 mM KCl, 20 mM Tris-HCl (pH 8), 0.1 mM MgCl₂)
Buffer 2: 100 mM NaCl, 50 mM Tris-HCl (pH 8), 0.5 mM MgCl₂

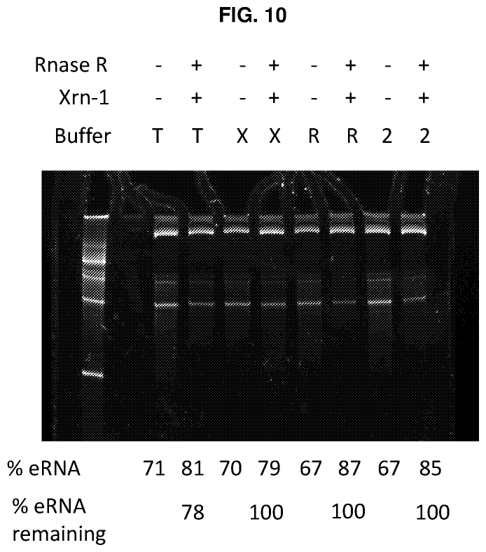

As controls, 2.5 μg of post-ligation polyribonucleotide mixture produced as described in Example 2, was also mixed in each buffer without exonuclease digestion (buffer controls). Each digestion was quenched with EDTA and analyzed by gel electrophoresis. The TBE-Urea gel was stained with SYBR Safe and imaged on an iBright imager (Invitrogen). The percentage of the total polyribonucleotides after digestion with the exonuclease that are circular polyribonucleotides (% circular), and the percentage of the circular polyribonucleotides that are remaining after digesting with the exonuclease compared the amount of circular polyribonucleotides that were present post-ligation (% eRNA remaining) were analyzed by Image Lab. FIG. 10 shows the results of the digestions with the combination of Xrn-1 and RNase R in various buffers and corresponding buffer controls (lane 1: NEB ssRNA ladder; lane 2: Terminator™ A buffer control (no exonucleases); lane 3: Terminator™ A buffer (Xrn-1+RNase R); lane 4: Xrn-1 buffer control (no exonuclease digestions); lane 5: Xrn-1 buffer (Xrn-1+RNase R); lane 6: RNase R buffer control (no exonuclease digestions); lane 7: RNase R buffer (Xrn-1+RNase R); lane 8: Buffer 2 control (no exonuclease digestions); lane 9: Buffer 2 (Xrn-1+RNase R). These results show that the circular polyribonucleotide in the post-ligation polyribonucleotide mixture was enriched to 87% after digestion with the combination of Xrn-1 and RNase R in RNase R Buffer 2 (lane 7). The circular polyribonucleotide in the post-ligation polyribonucleotide mixture was enriched to 85% after digestion with the combination of Xrn-1 and RNase R in Buffer 2 (lane 9).

### Example 9: In Vitro Expression of Gaussia Luciferase from Enriched Circular RNA

This example demonstrates that circular polyribonucleotide enriched using dual exonucleases expresses GLuc at levels comparable to circular polyribonucleotide purified without using dual exonucleases, using reverse-phase chromatography.

To generate the dual exonuclease, reverse phase purified sample, post-ligation polyribonucleotide mixture produced as described in Example 2, was digested concomitantly with 0.025 U of Terminator™ exonuclease per 1 μg of RNA and 0.5 U RNase R per 1 μg of RNA at 37° C. for 60 minutes. The digestion was quenched with EDTA and purified by reverse-phase chromatography. After chromatography and buffer exchange, the expression of the purified circular polyribonucleotide was tested by in vitro expression.

To generate the reverse phase purified sample, post-ligation polyribonucleotide mixture was purified by reverse-phase chromatography. After chromatography and buffer exchange, the expression of the purified circular polyribonucleotide was tested by in vitro expression.

HeLa cells (10,000) were transfected with 0.1 pmol of the purified circular polyribonucleotide using a transfection reagent (Lipofectamine MessengerMax (ThermoFisher, LMRNA015)). Supernatant was analyzed at 6 hours, 24 hours, 48 hours and 72 hours by measuring the activity of Gaussia Luciferase using a Gaussia Luciferase activity assay (Pierce Gaussia Luciferase Flash Assay Kit (16159)).

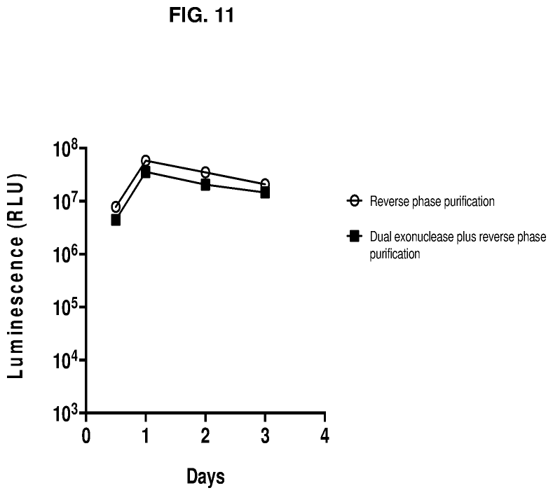

This Example demonstrated successful protein expression from enriched circular RNA (FIG. 11).

### Example 10: Selective Enrichment of Circular Polyribonucleotides of Various Molecular Weights

This example demonstrates the ability to selectively enrich circular polyribonucleotides using a combination of 5′ and 3′ exonucleases across a range of molecular weight sizes.

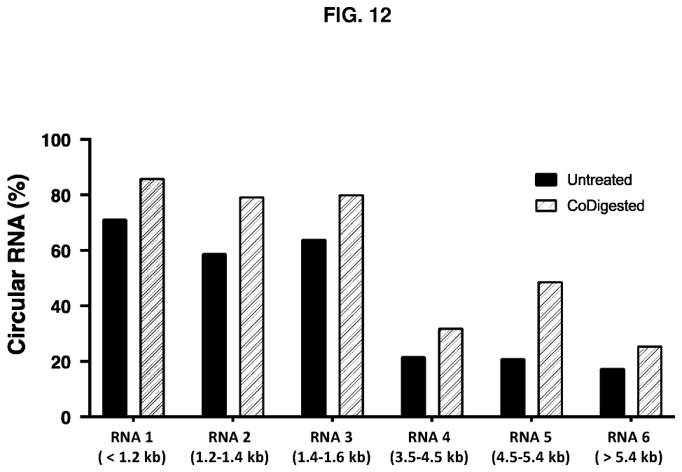

In this example, 150.0 μg of various polyribonucleotides were digested concomitantly with 0.625 U of RNase R and 0.0625 U of Terminator™ exonuclease per 1 μg of RNA at 37° C. for 60 minutes in Buffer 2. The molecular weights varied for each of the encoded polyribonucleotide ORFs evaluated. Each of RNA1-RNA6 are identical circular polyribonucleotides with a different sized expression sequence, increasing in molecular weights from left to right and ranging from less than 1.2 kb to greater than 5.4 kb. In each case, the digestion was quenched with EDTA and analyzed by anion-exchange high-performance liquid chromatography (AEX-HPLC). The percentage of the total polyribonucleotides after digestion with the exonucleases that are circular polyribonucleotides (% circular RNA) was compared to percentage of circular polyribonucleotides of the mixture prior to digestion. FIG. 12 shows the results of this example for selective enrichment of circular polyribonucleotides with a size of less than 1.2 kb, sizes ranging from 1.2-1.4 kb, 1.4-1.6 kb, 3.5-4.5 kb, 4.5-5.4 kb, and greater than 5.4 kb. The circular population of the total polyribonucleotides prior to digestion were determined to be 71.4, 59.0, 64.1, 21.9, 21.2, and 17.6 percent, respectively. After digestion with RNase R and Terminator™, the circular population of these same polyribonucleotides were found to be enriched to 86.0, 79.4, 80.2, 32.0, 48.9, and 25.7 percent, respectively.

### Example 11: Enrichment of Circular Polyribonucleotides Using Various Combinations of 5′ and 3′ Exonucleases

This example demonstrates the ability to selectively enrich the circular polyribonucleotide population using four combinations of 5′ and 3′ exonucleases

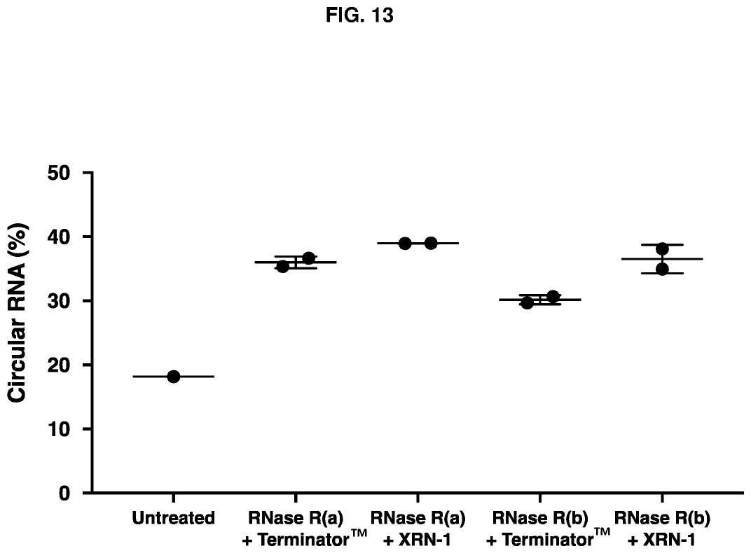

In this example, 150.0 μg of polyribonucleotides were digested concomitantly with, (i) 0.625 U of RNase R(a) and 0.0625 U of Terminator™; (ii) 0.625 U RNase R(a) and 0.0625 U of Xrn-1; (iii) 0.625 U RNase R(b) and 0.0625 U Terminator™; or (iv) 0.625 U RNase R(b) and 0.0625 U Xrn-1 exonucleases per 1 μg of RNA at 37° C. for 60 minutes. Each digestion was quenched with EDTA and analyzed by anion-exchange high performance liquid chromatography (AEX-HPLC). The percentage of the total polyribonucleotides after digestion with the exonucleases that are circular polyribonucleotides (% circular RNA) was compared to percentage of circular polyribonucleotides prior to digestion. The results of this example demonstrate that the selective enrichment of circular polyribonucleotide species can be achieved with various combinations of 5′ and 3′ exonucleases. FIG. 13 shows the results of the concomitant digestions using (i) RNase R(a) and Terminator™; (ii) RNase R(a) and Xrn-1; (iii) RNase R(b) and Terminator™; and (iv) RNase R(b) and Xrn-1. The percentage of circular polyribonucleotide in the post IVT polyribonucleotide mixture was just 18.2% prior to the co-digestion process. Following concomitant digestions, the percentage of circular polyribonucleotides were found to be enriched to (i) 36.0±0.91; (ii) 39.0±0.03; (iii) 30.2±0.70; and (iv) 36.5±2.23 percent, respectively.

1. A method of producing an enriched population of circular polyribonucleotides, the method comprising:
(a) providing a population of polyribonucleotides comprising circular polyribonucleotides and linear polyribonucleotides;
(b) digesting at least a portion of the linear polyribonucleotide with a 5′ exonuclease in an amount between 0.01 U and 0.2 U per 1 μg of polyribonucleotides; and
(c) digesting at least a portion of the linear polyribonucleotide with a 3′ exonuclease in an amount between 0.4 U and 4 U per 1 μg of polyribonucleotides;
thereby producing an enriched population of circular polyribonucleotides.

2. The method of claim 1, wherein the 5′ end of at least a portion of the linear polyribonucleotides comprises a monophosphate moiety.

3. The method of claim 1 of claim 2, wherein digesting step (b) is performed prior to step (c).

4. The method of claim 1 or claim 2, wherein the digesting step (b) is performed after step (c).

5. The method of claim 1 or claim 2, wherein digesting steps (b) and (c) are performed concomitantly.

6. The method of any one of claims 1-5, wherein the 5′ exonuclease is a 5′-phosphate dependent exonuclease.

7. The method of claim 6, wherein the 5′ exonuclease is Xrn-1.

8. The method of any one of claims 1-7, wherein the 3′ exonuclease is RNase R.

9. The method of any one of claims 1-7, wherein the 3′ exonuclease is Exonuclease T.

10. The method of anyone of claims 1-9, wherein digesting step (b) is performed for between 30 minutes and 90 minutes.

11. The method of any one of claims 1-10, wherein digesting step (c) is performed for between 30 minutes and 90 minutes.

12. The method of any one of claim 1-11, wherein the digesting step (b) is performed at a temperature of about 37° C.

13. The method of any one of claim 1-12, wherein the digesting step (c) is performed at a temperature of about 37° C.

14. The method of any one of claims 1-13, wherein digesting steps (b) and (c) are carried out in a digesting buffer comprising Mg²⁺.

15. The method of claim 14, wherein the Mg²⁺ in the digesting buffer has a concentration between 0.05 mM and 1 mM.

16. The method of any one claims 1-15, wherein digesting steps (b) and (c) are carried out in a digesting buffer comprising dithiothreitol.

17. The method of claim 16, wherein the dithiothreitol has a concentration of between 0.1 mM and 5 mM.

18. The method of any one of claims 1-17, wherein the 5′ exonuclease of step (b) is in an amount between 0.012 U an 0.1 U per 1 μg of polyribonucleotides.

19. The method of claim 18, wherein the 5′ exonuclease of step (b) is in an amount of 0.025 U per 1 μg of polyribonucleotides.

20. The method of any one of claims 1-19, wherein the 3′ exonuclease of step (c) is in an amount between 0.4 U and 2 U per 1 μg of polyribonucleotides.

21. The method of claim 20, wherein the 3′ exonuclease of step (c) is in an amount of 0.5 U per 1 μg of polyribonucleotides.

22. The method of any one of claims 1-21, wherein, following digesting steps (b) and (c), the percent (w/w) of the circular polyribonucleotides is between 40% and 95% of the total polynucleotides.

23. The method of claim 22, wherein, following digesting steps (b) and (c) the percent (w/w) of the circular polyribonucleotides is between 60% and 90% of the total polynucleotides.

24. The method of claim 23, wherein, following digesting steps (b) and (c), the percent (w/w) of the circular polyribonucleotides is between 70% and 90% of the total nucleotides.

25. The method of any one of claims 1-24, wherein, following digesting steps (b) and (c) the overall percent yield (w/w) of circular polyribonucleotide is between 70% and 100%.

26. A method of producing an enriched population of circular polyribonucleotides, the method comprising:
(a) providing a population of polyribonucleotides comprising circular polyribonucleotides and linear polyribonucleotides;
(b) digesting at least a portion of the linear polyribonucleotide with a 5′ exonuclease, and
(c) digesting at least a portion of the linear polyribonucleotide with a 3′ exonuclease,
wherein digesting steps (b) and (c) are performed in a digesting buffer comprising between 0.05 mM and 1 mM Mg²⁺ and/or between 0.1 mM and 5 mM dithiothreitol;
thereby producing an enriched population of circular polyribonucleotides.

27. The method of claim 26, wherein digesting step (b) is performed prior to step (c).

28. The method of claim 26, wherein the digesting step (b) is performed after step (c).

29. The method of claim 26, wherein digesting steps (b) and (c) are performed concomitantly.

30. The method of any one of claims 26-29, wherein the 5′ exonuclease in an amount between 0.01 U and 0.2 U per 1 μg of polyribonucleotides.

31. The method of any one of claims 26-30, wherein the 3′ exonuclease in an amount between 0.4 U and 4 U per 1 μg of polyribonucleotides.

32. The method of any one of claims 26-31, wherein the 5′ end of at least a portion of the linear polyribonucleotides comprises a monophosphate moiety.

33. The method of any one of claims 26-32, wherein the 5′ exonuclease is a 5′-phosphate dependent exonuclease.

34. The method of claim 33, wherein the 5′ exonuclease is Xrn-1.

35. The method of any one of claims 26-34, wherein the 3′ exonuclease is RNase R.

36. The method of any one of claims 26-34, wherein the 3′ exonuclease is Exonuclease T.

37. The method of anyone of claims 26-36, wherein digesting step (b) is performed for between 30 minutes and 90 minutes.

38. The method of any one of claims 26-37, wherein digesting step (c) is performed for between 30 minutes and 90 minutes.

39. The method of any one of claim 26-38, wherein the digesting step (b) is performed at a temperature of about 37° C.

40. The method of any one of claim 26-39, wherein the digesting step (c) is performed at a temperature of about 37° C.

41. The method of any one of claims 26-40, wherein the 5′ exonuclease of step (b) is in an amount between 0.012 U an 0.1 U per 1 μg of polyribonucleotides.

42. The method of claim 41, wherein the 5′ exonuclease of step (b) is in an amount of 0.025 U per 1 μg of polyribonucleotides.

43. The method of any one of claims 26-42, wherein the 3′ exonuclease of step (c) is in an amount between 0.4 U and 2 U per 1 μg of polyribonucleotides.

44. The method of claim 43, wherein the 3′ exonuclease of step (c) is in an amount of 0.5 U per 1 μg of polyribonucleotides.

45. The method of any one of claims 26-44, wherein, following digesting steps (b) and (c), the percent (w/w) of the circular polyribonucleotides is between 40% and 95% of the total polynucleotides.

46. The method of claim 45, wherein, following digesting steps (b) and (c) the percent (w/w) of the circular polyribonucleotides is between 60% and 90% of the total polynucleotides.

47. The method of claim 46, wherein, following digesting steps (b) and (c), the percent (w/w) of the circular polyribonucleotides is between 70% and 90% of the total nucleotides.

48. The method of any one of claims 26-47, wherein, following digesting steps (b) and (c) the overall percent yield (w/w) of circular polyribonucleotide is between 70% and 100%.
